(19) United States
(12) Patent Application Publication
(10) Pub. No.: US 2007/0184021 A1
(43) Pub. Date: Aug. 9, 2007

(54) Use of caspase inhibitors as a therapeutic agent against radiation-induced injury

(76) Inventors: Daohong Zhou, Mt. Pleasant, SC (US); Aimin Meng, Beijing (CN)

Correspondence Address: FULBRIGHT & JAWORSKI L.L.P., 600 CONGRESS AVE., SUITE 2400, AUSTIN, TX, 78701, US

(21) Appl. No.: 10/529,661

(22) PCT Filed: Sep. 30, 2003
(86) PCT No.: PCT/US03/30847

§ 371 Date: Apr. 3, 2007

Related U.S. Application Data
(60) Provisional application No. 60/415,182, filed on Oct. 1, 2002.

(51) Int. Cl. A61K 38/20; A61K 48/00; A61K 38/17

(52) U.S. Cl. 424/85.2; 424/93.2; 514/12

(57) ABSTRACT

The present invention provides methods for the treatment of radiation-induced injury by providing to a subject a caspase inhibitor. The caspase inhibitor may be provided before or after the radiation injury, and further may be provided in combination with a second caspase inhibitor, an anti-apoptotic molecule, a radioprotectant, a cytokine or a growth factor.

This application claims the benefit of U.S. Provisional Application No. 60/415,182, filed Oct. 1, 2002, the entire text of which, is specifically incorporated by reference.

### BACKGROUND OF THE INVENTION

The government owns rights in the present invention pursuant to grant number CA78688 and CA86860 from the National Institutes of Health.

1. Field of the Invention

The present invention relates generally to the fields of radiation biology and medicine. More particularly, it concerns methods for the prevention and treatment of radiation injury comprising administration of caspase inhibitors to subjects suffering from or at risk of radiation injury.

2. Description of Related Art

Exposure to ionizing radiation (IR) due to radiotherapy and nuclear accident causes myelosuppression in a dose-dependent manner (Mauch et al., 1995). The myelosuppression is the major dose-limiting factor of radiotherapy for cancer and the primary cause of death after accidental exposure to a high dose of radiation (Mauch et al., 1995). An acute and transient myelosuppression may result from IR-induced damage to the rapidly proliferating hematopoietic progenitors and their more mature progenies that are highly sensitive to IR. Conversely, a persistent myelosuppression or bone marrow (BM) failure following IR indicates an injury of hematopoietic stem cells (HSC) that have the ability of self-renewing and producing all the progenies of different hematopoietic lineages.

Despite the fact that HSC are largely a non-proliferating population, they are extremely radiosensitive. The mechanisms by which IR induces HSC injury remain obscure, because the paucity of HSC makes the study of them relatively difficult. There is indirect evidence suggesting that IR may damage HSC by induction of apoptosis. First, IR is a potent inducer of apoptosis in many different types of cells (Harms-Ringdahl et al., 1996). Second, overexpression of bcl-2, an anti-apoptotic protein, throughout the hematopoietic compartment protects mice against IR-induced hematopoietic failure and death (Domen et al., 1998). HSC isolated from bcl-2 transgenic mice are more resistant to IR-induced damage in vitro (Domen et al., 1998). In contrast, bcl-2 deficiency sensitizes murine HSC to IR (Hoyes et al., 2000). In addition, the HSC from p53- or Fas-deficient mice are less sensitive to IR than those from wild-type mice (Cui et al., 1995; Hirabayashi et al., 1997; Nagafuji et al., 1996; Perkins et al., 1987). However, there is no direct evidence to demonstrate that HSC respond to IR by apoptosis.

Moreover, the damage of IR to a cell is not limited to the induction of apoptosis, but also includes the induction of necrosis and senescence (Di Leonardo et al., 1994; Seidita et al., 2000). For example, exposure of human fibroblasts to IR causes clonogenic cell deletion by induction of premature senescence (Di Leonardo et al., 1994; Seidita et al., 2000). Although DNA damage is likely the primary cause for IR-induced apoptosis, necrosis and senescence, the signal transducing processes originated from IR-induced DNA damage leading to these diverse cellular responses may be different. For example, the induction of Bax and other proapoptotic proteins by p53 may be responsible for IR-induced apoptosis (Norbury and Hickson, 2001; Shen and White, 2001), while p53-mediated induction of p21 and p16 by IR may cause permanent cell cycle arrest or senescence (Di Leonardo et al., 1994; Seidita et al., 2000). Thus, in order to effectively protect HSC from IR, it is important to determine the precise mechanism by which IR causes HSC damage. Following such a determination, one will gain a better understanding of the cellular and molecular mechanisms for IR-induced myelosuppression, hopefully leading to the discovery of more effective ways to circumvent IR BM toxicity.

### SUMMARY OF THE INVENTION

Therefore, in accordance with the present invention, there is provided a method of inhibiting apoptosis in a hematopoietic stem cell (HSC) comprising contacting the cell with a caspase inhibitor in an amount sufficient to inhibit apoptosis in the cell. The apoptosis may be induced by ionizing radiation. The HSC may be contacted with the ionizing radiation before the caspase inhibitor, for example about 4 h prior to receiving the caspase inhibitor. The HSC may be contacted with the ionizing radiation after the caspase inhibitor, for example, about 2 h after receiving the caspase inhibitor. The caspase inhibitor may be contacted with the HSC more than one time. The caspase inhibitor may be administered both prior to and after ionizing radiation is contacted with the HSC. The caspase inhibitor may be z-VAD, BocD, LY333531, casputin, Ac -DQMD-CHO, CV-1013, VX-799, Ac-YVAD-CMK, IDN-5370, IDN-6556, IDN-6734, IDN-1965, IDN-1529, z-VAD-fmk, z-DEVD-cmk, Ac-YVAD-fmk, z-Asp-Ch2-DCB, Ac-IETD, Ac-VDVAD, Ac-DQMD, Ac-LEHD, or Ac-VEID. The HSC may be contacted with a second caspase inhibitor, an anti-apoptotic molecule (a p53 inhibitor or an anti-apoptotic protein, such as Bcl-X_{L}, Bcl-2, c-IAP1, c-IAP2, and XIAP), a radioprotectant (amifostine, vitamin E, vitamin C, selenium, melatonin, 5-androstenediol, cucumin, α-phenyl-tert-butylnitrone, a flavinoid, or a nitroxide), a cytokine (IL-1, IL-2, IL-3, IL-4, IL-5, IL-6, IL-7), or a growth factor (Flt3 ligand, c-Kit ligand, M-CSF, GM-CSF, G-CSF, VEGF, erythropoietin, leukemia inhibitory factor). The anti-apoptotic protein, cytokine or growth factor may be expressed in the HSC from a recombinant expression vector. The expression vector may be a viral vector or a non-viral vector.

In a further embodiment, there is provided a method of inhibiting radiation-induced injury in a subject comprising administering to the subject a caspase inhibitor in an amount sufficient to inhibit radiation-induced injury. The caspase inhibitor may be administered orally or by injection. The caspase inhibitor may be z-VAD, BocD, LY333531, casputin, Ac-DQMD -CHO, CV-1013, VX-799, Ac-YVAD-CMK, IDN-5370 IDN-6556, IDN-6734, IDN-1965, IDN-1529, z-VAD-fmk, z-DEVD-cmk, Ac-YVAD-fmk, z-Asp-Ch2-DCB, Ac-IETD, Ac-VDVAD, Ac-DQMD, Ac-LEHD, or Ac-VEID. The method may further comprise administering to the subject a second agent selected from the group consisting of a second caspase inhibitor, an anti-apoptotic molecule (a p53 inhibitor or an anti-apoptotic protein, such as Bcl-X_{L}, Bcl-2, c-IAP1, c-IAP2, and XIAP), a radioprotectant (amifostine, vitamin E, vitamin C, selenium, melatonin, 5-androstenediol, cucumin, α-phenyl-tert-butylnitrone, a flavinoid, or a nitroxide), a cytokine (IL-1, IL-2, IL-3, IL-4, IL-5, IL-6, IL-7), a growth factor (Flt3 ligand, c-Kit ligand, M-CSF, GM-CSF, G-CSF, VEGF, erythropoietin, leukemia inhibitory factor). The anti-apoptotic protein, cytokine or growth factor may be expressed from a recombinant expression vector encoding the anti-apoptotic protein and an HSC-selective promoter. The caspase inhibitor may be provided prior to exposure to radiation or following exposure to radiation, for example, about 4 h or less following exposure to radiation. The caspase inhibitor may be administered more than once. The caspase inhibitor may be provided via continuous infusion.

In yet another embodiment, there is provided a method of screening a caspase inhibitor for its ability to inhibit radiation-induced injury comprising (a) providing a hematopoietic stem cell (HSC); (b) contacting the HSC with a dose of ionizing radiation sufficient to induce apoptosis in the HSC; (c) contacting the HSC with the caspase inhibitor; and (d) assessing one or more apoptotic characteristics in the HSC, wherein a reduction in the number or extent of apoptotic characteristics in the HSC, as compared to an HSC not treated with the caspase inhibitor, identifies the caspase inhibitor as an inhibitor of radiation-induced injury. The method may comprise the use of multiple HSCs, and assessing comprises measuring the number of the HSCs undergoing apoptosis. Assessing may comprise TUNEL assay, Annexin V-7AAD or PI staining, sub G0/1 cell analysis, caspase activity assay, or flow cytometry that can discriminate between Lin⁻ Sca1⁺ c-kit⁺, Lin⁻ Sca1⁻ c-kit⁺, Lin⁻ Sca1⁺ c-kit⁻, and Lin⁻ Sca1⁻ c-kit⁻ cells. At least steps (b) and (c) may be performed in vivo. HSC may be isolated and at least steps (b) and (c) performed in vitro. The characteristics of apoptosis can include Annexin-V staining, caspase activation, DNA fragmentation. The method may further comprise contacting the HSC with a second agent that is a radioprotectant. The method may further comprise assessing one or more apoptotic characteristics in an HSC not treated with the caspase inhibitor.

In still yet another embodiment, there is provided a composition comprising a radioprotectant and a second agent selected from the group consisting of an anti-apoptotic molecule (a p53 inhibitor or an anti-apoptotic protein, such as Bcl-X_{L}, Bcl-2, c-IAP1, c-LAP2, and XIAP), a radioprotectant (amifostine, vitamin E, vitamin C, selenium, melatonin, 5-androstenediol, cucumin, α-phenyl-tert-butylnitrone, a flavinoid, or a nitroxide), a cytokine (IL-1, IL-2, IL-3, IL-4, IL-5, IL-6, IL-7), or a growth factor (Flt3 ligand, c-Kit ligand, M-CSF, GM -CSF, G-CSF, VEGF, erythropoietin, leukemia inhibitory factor).

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Phenotypic analysis of Lin⁻ cells with or without exposure to IR. Lin⁻ cells (1×10⁶/ml) were non-irradiated (Control) or exposed to 4 Gy IR. After 6 or 18 h incubation, the cells were stained with Sca-1-PE and c-kit-APC antibodies and a minimum of 150,000 cells was analyzed by flow cytometry. The data presented are an example of the analysis.

FIG. 2A-B. Analysis of IR-induced apoptosis and/or necrosis in Lin⁻ Sca-1⁺ c-kit⁺ and Lin⁻ Sca-1⁻ c-kit⁺ cells by flow cytometry. Lin− cells (1×10⁶/ml) were non-irradiated (Control) or exposed to 4 Gy IR. After 6 or 18 h incubation, the cells were stained with Sca-1-PE and c-kit-APC antibodies and then with annexin V-FITC and 7-AAD. A minimum of 150,000 cells was analyzed by flow cytometry. The data presented are an example of the analysis. The early and late stage apoptotic cells are annexin V⁺ and annexin V⁺/7-AAD⁺, respectively. The necrotic cells are 7-AAD⁺, whereas the live cells are double negative (annexin V⁻/7-AAD⁻).

FIGS. 3A-B. IR induces apoptosis in Lin⁻ Sca-1⁺ c-kit⁺ and Lin⁻ Sca-1⁻ c-kit⁺ cells. Lin− cells (1×10⁶/ml) were non-irradiated (Control) or exposed to 4 Gy IR. After 6 or 18 h incubation, the cells were stained with Sca-1-PE and c-kit-APC antibodies and then with annexin V-FITC and 7-AAD. A minimum of 150,000 cells was analyzed by flow cytometry. The percentage of annexin V⁺ and annexin V⁺/7-AAD⁺ cells were added together to represent the total numbers of cells undergoing apoptosis. The data are presented as mean ±SD of triplicates. Similar results were observed in two additional independent experiments. *p<0.001 vs Control.

FIGS. 4A-B. Analysis of the effect of z-VAD on IR-induced apoptosis and/or necrosis in Lin⁻ Sca-1⁺ c-kit⁺ and Lin⁻ Sca-1⁻ c-kit⁺ cells by flow cytometry. Lin− cells (1×10⁶/ml) were pre-incubated with 0.2% DMSO (Vehicle) or 100 μM z-VAD (in DMSO) for 1 h prior to exposure to 4 Gy IR or un-irradiated (Control). After 6 or 18 h incubation, the cells were stained with Sca-1-PE and c-kit-APC antibodies and then with annexin V-FITC and 7-AAD. A minimum of 150,000 cells was analyzed by flow cytometry. The data presented are an example of the analysis.

FIGS. 5A-B. VAD inhibits IR-induced apoptosis in Lin⁻ Sca-1⁺ c-kit⁺ and Lin⁻ Sca-1⁻ c-kit⁺ Cells. Lin− cells (1×10⁶/ml) were pre-incubated with 0.2% DMSO (Vehicle) or 100 μM z-VAD (in DMSO) for 1 h prior to exposure to 4 Gy IR or un-irradiated (Control). After 6 or 18 h incubation, the cells were stained with Sca-1-PE and c-kit-APC antibodies and then with annexin V-FITC and 7-AAD. A minimum of 150,000 cells was analyzed by flow cytometry The percentage of annexin V⁺ and annexin V⁺/7-AAD⁺ cells were added together to represent the total numbers of cells undergoing apoptosis. The data are presented as mean ±SD of triplicates. Similar results were observed in two additional independent experiments. FIG. 5A, p<0.001 vs Vehicle/Control; FIG. 5B, p<0.001 vs Vehicle/IR

FIGS. 6A-B. VAD inhibits IR-induced decrease in the numbers of Lin⁻ Sca-1⁺ c-kit⁺ and Lin⁻ Sca-1⁻ c-kit⁺ sells. Lin− cells (1×10⁶/ml) were pre-incubated with 0.2% DMSO (vehicle) or 100 μM z-VAD for 1 h prior to exposure to 4 Gy IR or un-irradiated (control). After 6 or 18 h incubation, the cells were stained with Sca-1-PE and c-kit-APC antibodies and then with annexin V-FITC and 7-AAD. A minimum of 150,000 cells was analyzed by flow cytometry. The numbers of each phenotype of Lin⁻ cells were calculated by multiplication of the total numbers of cells harvested with the percentage of each phenotype of Lin⁻ cells determined by flow cytometric analysis, and expressed as a percentage of the numbers of un-irradiated control cells. The data are presented as mean ±SD of triplicates. Similar results were observed in two additional independent experiments. *p<0.001 vs Vehicle/IR.

FIGS. 7A-B. Post-IR treatment with Z-VAD inhibits IR-induced apoptosis in Lin⁻ Sca-1⁺ c-kit⁺ and Lin⁻ Sca-1⁻ c-kit⁺ Cells. Lin− cells (1×10⁶/ml) were pre-incubated with 0.2% DMSO (IR) or 100 μM z-VAD for 1 h (−1 h) prior to IR exposure, or they were treated with z-VAD immediately before (0 h) or 30 min (0.5 h), 1 h, 2 h or 4 h after IR exposure. Cells treated with vehicle but non-irradiated were used as control (C). After 6 or 18 h incubation, the cells were stained with Sca-1-PE and c-kit-APC antibodies and then with annexin V-FITC and 7-AAD. A minimum of 150,000 cells was analyzed by flow cytometry. The percentage of annexin V⁺ and annexin V⁺/7-AAD⁺ cells were added together to represent the total numbers of cells undergoing apoptosis. The data are presented as mean ±SD of triplicates. FIG. 7A, p<0.001 vs control (C); FIG. 7B, p<0.001 vs IR.

FIGS. 8A-B. z-VAD partially protects HSC from IR-induced suppression of hematopoietic function. BM cells were pre-incubated with vehicle or 100 μM z-VAD for 1 h and then were exposed to 4 Gy IR. BM cells without IR were used as control. Six hours after the IR, the cells were overlaid on FBMD-1 stromal cell layers. The frequency of CAFC was determined on 7, 14, 21, 28 and 35 days after IR. FIG. 8A—Frequency of CAFC. FIG. 8B—Survival fraction of CAFC as compared to that of control cells. The data presented are mean +/− SEM (n=3 individual mice/groups). Vehicle/IR vs z-VAD/IR, p<0.01 by ANOVA.

FIG. 9. z-VAD protects mice from IR-induced lethality. Male C57BL/6 mice were given two i.p. injections of vehicle (0.25 ml PBS with 0.2% DMSO) or z-VAD (6 mg/kg) at one hour prior to and 5 h after exposure to a lethal dose of IR (10.5 Gy). The survival of these mice was monitored daily up to 24 days after exposure to IR. n=6 mice/group).

### DESCRIPTION OF ILLUSTRATIVE EMBODIMENTS

In light of the events of Sep. 11, 2001, many anti-terrorist experts and government officials believe that the prospect of a nuclear terrorist attack against the United States is a virtual certainty. For example, terrorists may have the ability of making and using a “dirty bomb”—that disperses radioactive material using a conventional explosive. Detonation of a dirty bomb can release deadly radioactive particles into the atmosphere contaminating the inhabitants of targeted cities and farmlands. There is also the possibility that terrorist groups could target nuclear power plants. Attacks on nuclear power plants could come from hijacked airplanes, a truck used as a bomb, or direct attack on the plant. The attacks could result in release of a large amount of radioactive materials from the plant. Thousands of people could be potentially exposed to radiation and affected, leading to long- and short-term health consequences and possible death. These increased nuclear threats place not only the military personnel but also the general public at great risks of exposure to ionizing radiation (IR) exposure. Thus, in addition to the continuing need for protection from IR in the medical field, as discussed above, there is an urgent need to develop new strategies to combat the nuclear terrorism.

An ideal radioprotectant can be used to protect the military personnel and the general public in the event of a nuclear terrorist attack, warfare or accident, as well as provide benefits in a more controlled clinical setting. The characteristics of such a drug would be: (1) highly protective against IR-induced tissue damage; (2) relatively stable and easily administered (oral administration preferred); (3) low in toxicity; and (4) effective, even if used after IR exposure. Conventional radioprotectants, represented best by Amifostine, can be highly protective if they are administered i.p. or i.v. prior to radiation exposure, but not by oral route administration. However, they offer little protection if they are used after exposure to IR. Thus, these conventional radioprotectants have minimal utility as therapeutic agents for post-radiation rescue therapy following a nuclear terrorist attack, warfare or accident.

I. The Present Invention

A limiting factor in the development of radioprotective agents is the lack of information on the precise mechanism of injury. One hypothesis is that whole body irradiation causes persistent myelosuppression and hematopoietic by induction of bone marrow (BM) cell apoptosis, particularly in the hematopoietic stem cell (HSC) compartment (Mauch et al., 1995; Domen et al., 1998; Hoyes et al., 2000; Cui et al., 1995; Hirabayashi et al., 1997; Nagafuji et al., 1996). However, the scarcity of HSC has made the study of HSC apoptosis difficult. Thus, up to now only indirect evidence is available to suggest that IR may induce HSC apoptosis ) Domen et al., 1998; Hoyes et al., 2000; Cui et al., 1995; Hirabayashi et al., 1997; Nagafuji et al., 1996).

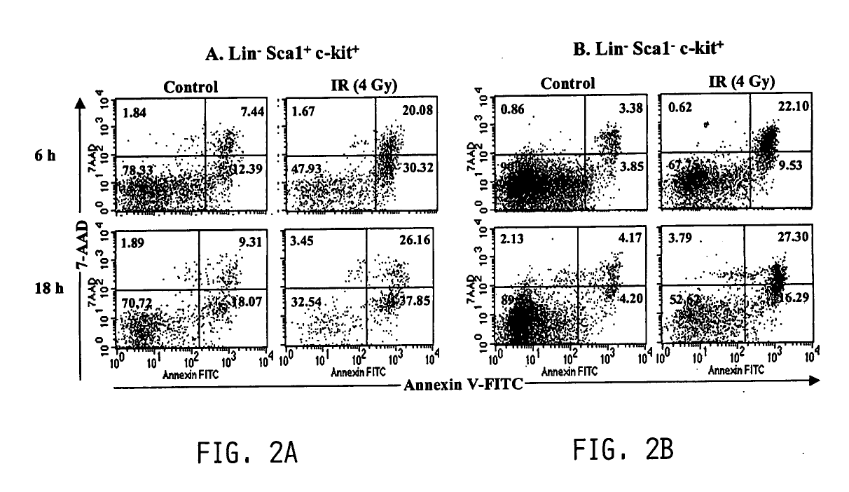

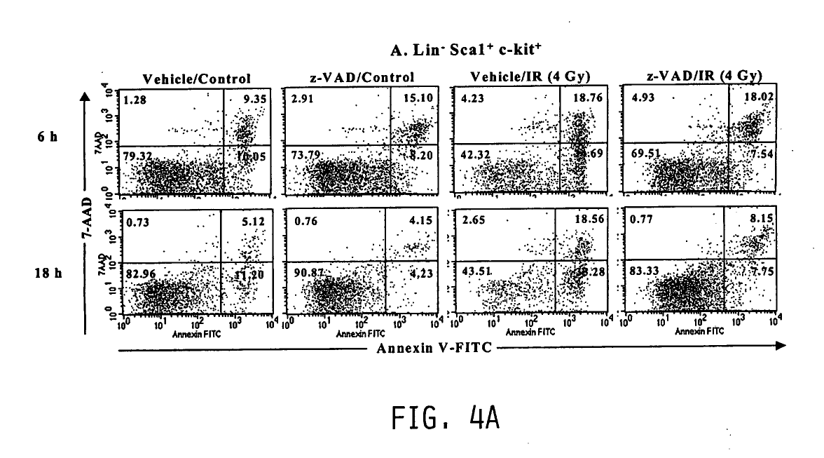

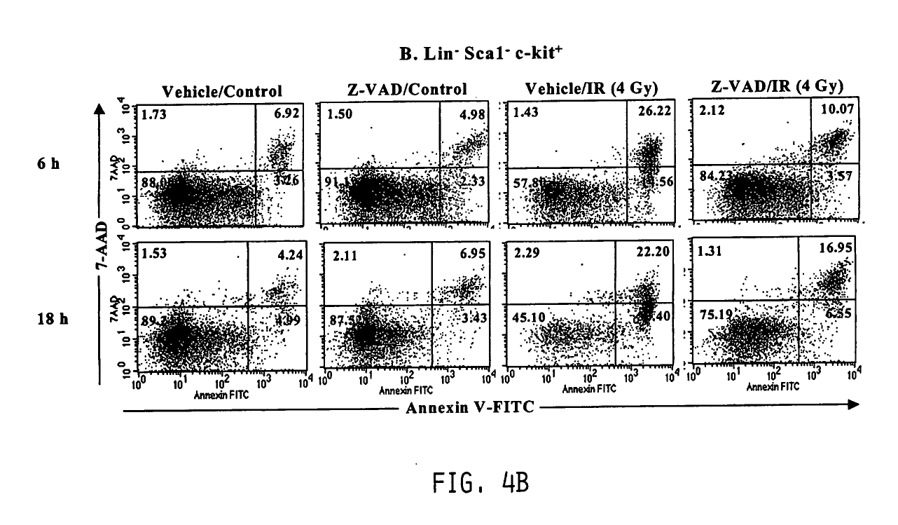

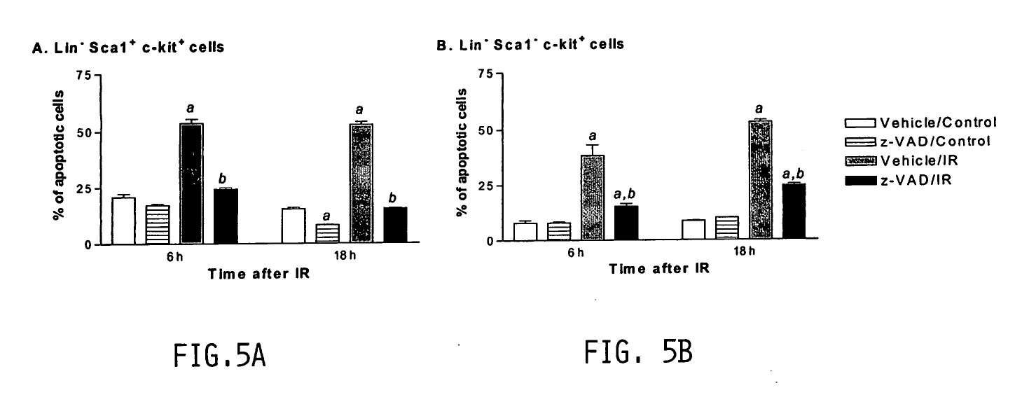

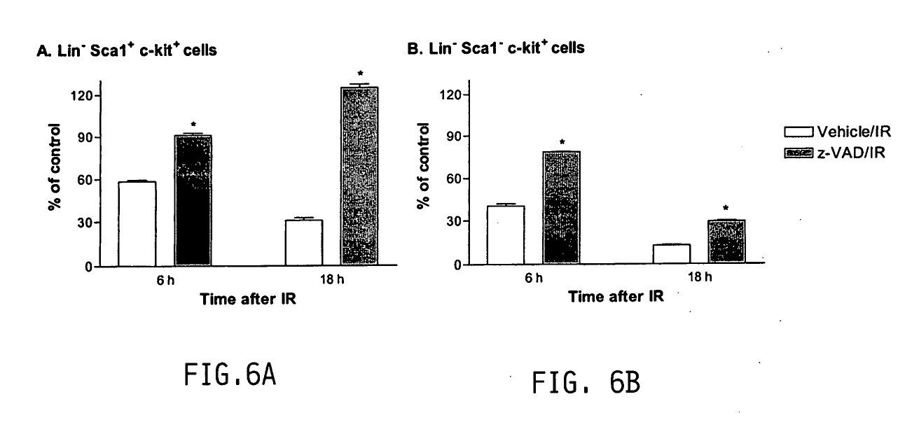

Using isolated Lin⁻ cells from mouse BM in combination with flow cytometric analysis of annexin V and/or 7-AAD staining, the inventors directly assessed whether IR induces apoptosis and/or necrosis in Lin⁻ Sca1⁺ c-kit⁺ cells that are highly enriched HSC (Hasper et al., 2000; Okada et al., 1992). For the first time, the inventors were able to demonstrate that IR mainly induced HSC cell death by apoptosis, but not by necrosis (FIGS. 2A&B). This finding was further supported by the observation that inhibition of caspase activities with z-VAD abrogated IR-induced apoptosis in Lin⁻ Sca1⁺ c-kit⁺ cells (FIGS. 4A&B and 5A&B). Moreover, z-VAD treatment also completely prevented Lin⁻ Sca1⁺ c-kit⁺ cells from IR-induced decrease, suggesting that apoptotic cell death is mainly responsible for the reduction in the numbers of HSC after IR (FIGS. 6A&B).

The finding that IR induces HSC damage by apoptosis provides a new mechanism of therapeutic intervention to protect BM against IR. One such approach is to use z-VAD and other caspase inhibitors to inhibit HSC apoptosis. Since activation of caspases usually occurs down stream after a cell receives an apoptotic insult (Earnshaw et al., 1999; Pruschy et al., 2001), this leaves some time for a post IR treatment using a caspase inhibitor. This suggestion is well supported by the finding that the delayed z-VAD treatment up to 4 hours post-IR remained effective at inhibiting IR-induced apoptosis in HSC and progenitors. Moreover, z-VAD treatment did not lead to a switch from IR-induced apoptosis to necrosis in Lin⁻ Sca1⁺ c-kit⁺ as was shown in some other cells (Los et al., 2002; Sane and Bertrand, 1999), but completely protected these cells from IR-induced reduction. Therefore, in an event of an accidental or terroristic exposure to IR, caspase inhibitors will be extremely useful as a post-radiation therapeutic agent to protect the hematopoietic system against IR-induced damage.

H. Hematopoietic Stem Cells

Hematopoietic stem cells (HSCs) are the rare cells that give rise to all the lineages of the blood. They are a largely non-proliferating population, yet are extremely sensitive to radiation injury. While the phenotypic and functional characteristics of HSCs have been well studied, the mechanisms that regulate their growth remain poorly understood, at least in part because they are so scarce that studies with HSCs are difficult. It has been shown that Lin⁻ Sca1⁺ c-kit⁺ cells are enriched in HSCs that have the ability to give rise to long term multilineage reconstitution (Okada et al., 1992). Lin⁻ Sca1⁻ c-kit⁺ cells are progenitors that are only capable of short term hematopoietic reconstitution (Okada et al., 1992). Conversely, Lin⁻ Sca1⁺ c-kit⁻ and Lin⁻ Sca1⁻ c-kit⁻ cells are devoid of both HSC and progenitors (Okada et al., 1992). Based on these criteria, one can employ flow cytometry to separate these cells, as well as study them.

III. Radiation Injury

The absorption of a sufficient amount of energy from radiation can lead to ejection of one or more orbital elections from biological molecules. This process is called ionization, and the type of radiation giving rise to such effects is said to be ionizing radiation (IR). The common types of IR include electromagnetic (X- and γ-rays) and particulate (protons, neutrons and α-particles) radiation. When these effects occur in the cells and tissues of a living organism, radiation injury ensues.

Within a few hours or days after exposure to a sufficient dose of IR (from a medical procedure, accident or terrorist attack), a series of characteristic clinical sequelae termed the acute radiation syndrome (ARS) appears (Granier and Gambini, 1990; Young, 1987). The ARS consists of three phases. The prodromal phase develops shortly after IR exposure and lasts for a few hours. The prodromal syndrome includes nausea, vomiting, diarrhea, a feeling of malaise, and fatigue. This is followed by an incubation period or latent stage that can last for days or weeks. The latent phase is followed by the manifestation of illness associated with various degrees and specificity of tissue injury. Significant tissue injuries exhibited in the ARS are dose-dependent ranging from hematopoietic, to gastrointestinal, and to neurovascular syndrome as a function of the dose of IR exposed. Additional injury can occur in gonads, lungs and skin. In general, hematopoietic failure is the primary cause of death after exposure to a high dose of radiation because the bone marrow (BM) is extremely sensitive to IR (Mauch et al., 1994; Zucali, 1994). Thus, protecting BM from IR-induced damage is critical for the survival of IR patients. Late effects of IR are common in individuals exposed to a low dose of IR and high dose IR survivors (D)evine and Chaput, 1987). These include the somatic and genetic effects. Somatic alterations include cataracts, changes in growth and development, non-specific shortening of life span, and various malignancies. Genetic alterations include mutations and chromosome aberrations that manifest in the descendants of an exposed individual.

The inventors have yet to determine precise molecular trigger by which IR-mediated HSC apoptosis is induced. It has been shown that exposure of cells to IR induces DNA damage and activates ATM (Norbury and Hickson, 2001; Shen and White, 2001). Activation of ATM in turn causes accumulation and activation of p53. Induction of pro-apoptotic proteins, such as Bax, by p53 may lead to HSC apoptosis (Norbury and Hickson, 2001; Shen and White, 2001). Alternatively, IR may induce Fas and/or FasL expression by HSC or other cells in BM (Nagafuji et al., 1996). Interaction of Fas and FasL can initiate apoptotic process in HSC (Nagafuji et al., 1996). Indeed, it has been reported that HSC from the p53-deficient or Fas-defective MRL/1pr mice appear more resistant to IR, as compared to the cells from wild type mice (Cui et al., 1995; Hirabayashi et al., 1997; Perkins et al., 1987). Either way, the activation of caspases is necessary for both p53 and Fas-FasL to induce apoptosis in HSC (Earnshaw et al., 1999; Pruschy et al., 2001). This may explain why z-VAD is effective in inhibiting IR-induced apoptosis in HSC. However, z-VAD is a non-specific caspase inhibitor that has the ability to inhibit multiple forms of caspases (Naito et al., 1997; Susin et al., 1999).

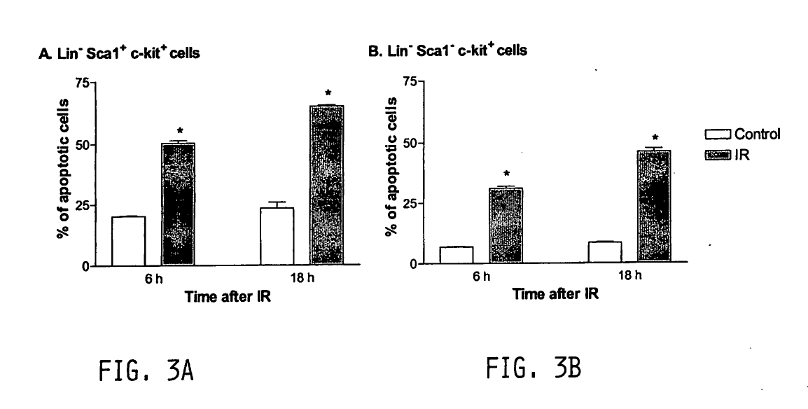

As it would be expected that the committed hematopoietic progenitors are usually more sensitive to IR than HSC (Down et al., 1995; Ploemacher et al., 1992), exposure of Lin⁻ cells to IR caused a greater decrease in the numbers of Lin⁻ Sca1⁻ c-kit⁺ cells than that in Lin⁻ Sca1⁺ c-kit⁺ cells (Table 1). Similarly, induction of apoptosis, but not necrosis, was seen in Lin⁻ Sca1⁻ c-kit⁺ cells after IR, suggesting that IR also induces apoptosis in progenitors. However, the percentage of apoptotic cells in Lin⁻ Sca1⁻ c-kit⁺ cells was lower than that in Lin⁻ Sca1⁺ c-kit⁺ cells after exposure to IR (FIGS. 3A&B). Moreover, z-VAD treatment was less effective in protecting Lin⁻ Sca1⁻ c-kit⁺ cells from IR-induced reduction than Lin⁻ Sca1⁺ c-kit⁺ cells (FIGS. 6A&B and 7A&B). These findings suggest that, unlike with HSC, the induction of apoptosis may be only partially responsible for IR-induced decrease in progenitors. Other factors, such as down regulation of c-kit expression that has been seen in Lin⁻ Sca1⁻ c-kit⁺ cells after 5-FU treatment (Randall and Weissman, 1997), may also contribute to IR-induced reduction in progenitors.

IV. Caspase Inhibitors

The finding that IR induces HSC damage by apoptosis permits new therapeutic interventions to protect these, and possibly other bone marrow cells, against IR. In particular, one of these approaches is to use caspase inhibitors to inhibit HSC apoptosis. Indeed, the inventors have shown the caspase inhibitor z-VAD not only inhibits IR-induced HSC apoptosis, but also preserves hematopoietic function as shown in in vitro cobblestone area-forming cell (CAFC) assays. More importantly, when z-VAD (6 mg/kg, i.p.) was administered to mice twice (one hour prior to and five hours after) during exposure to a lethal dose of IR (10.5 Gy), it protected half of these mice from IR-induced death, while the vehicle-treated mice died within 9 days after exposure to the same dose of IR. These findings provide compelling support for the use of caspase inhibitors to ameliorate IR-induced HSC and other tissue damage.

Since activation of caspases is a down-stream event occurring after a cell receives an apoptotic insult, sufficient time should be available to allow for post-IR treatment. This suggestion is strongly supported by the present studies demonstrating that even with a delay in z-VAD treatment for up to 4 h post-IR, inhibition of IR-induced apoptosis in HSC still was observed. Therefore, in the event of an accidental exposure to IR, such as a nuclear terrorist attack, caspase inhibitors provide important advantage over existing radioprotectants in that they can be administered following the IR insult. Thus, this identifies an extremely useful as a new class therapeutic agents in the protection of hematopoietic system and other tissues against IR-induced damage.

There are a large number of caspase inhibitors that can be employed according to the present invention. In addition to the exemplified z-VAD, other specific caspase inhibitors include BocD, LY333531, casputin, Ac-DQMD-CHO, CV-1013, VX-799, Ac-YVAD-CMK, IDN-5370 IDN-6556, IDN-6734, IDN-1965, IDN-1529, z-VAD-fmk, z-DEVD-cmk, Ac -YVAD-fmk, z-Asp-Ch2-DCB, Ac-IETD, Ac-VDVAD, Ac-DQMD, Ac-LEHD, and Ac-VEID.

Peptide and peptidyl inhibitors of ICE, a caspase, have been described. PCT patent applications WO 91/15577; WO 93/05071; WO 93/09135; WO 93/14777 and WO 93/16710; and EP 547 699. Such peptidyl inhibitors of ICE have been observed to block the production of mature IL-1β in a mouse model of inflammation (vide infra) and to suppress growth of leukemia cells in vitro (Estrov et al., 1994). Non-peptidyl compounds have also been reported to inhibit ICE in vitro. See PCT patent application WO 95/26958; U.S. Pat. No. 5,552,400; Dolle et al. (1996). Other caspase inhibitors can be found U.S. Pat. No. 6,426,413. Each of the foregoing references are thus incorporated by reference.

V. Conventional Radioprotective Agents

The majority of conventional radioprotectants are potent antioxidants (Giambarresi and Jacobs, 1987). Among these, Amifostine (or WR-2721) is the most effective. Amifostine is S-2-(3-aminopropyl-amino)ethylphosphorothioic acid, an analog of β-mercaptoethylamine (MEA) with a phosphate group on the sulfur and propylamino group on the nitrogen function. Amifostine functions as a pro-drug. Dephosphorylation of Amifostine exposes the sulfur group and releases the active free thiol compound WR-1065. Compared to other aminothiol compounds, Amifostine represents a significant improvement in effectiveness, potency and reduced toxicity and has a much higher therapeutic index than that for MEA or the active drug WR-1065 (Giambarresi and Jacobs, 1987; Poggi et al., 2001). Other known radioprotectants include vitamins (E and C), selenium, hormones (melatonin and 5-androstenediol), natural antioxidants derived from various plants (such as cucumin and flavonoids) and spin trapping agents (nitroxides and a-phenyl-tert-butylnitrone) (Giambarresi and Jacobs, 1987; Poggi et al., 2001; Weiss and Landauer, 2000). These compounds in general are less toxic than aminothiol compounds, but their effectiveness as radioprotectants remains to be established as compared to Amifostine.

At the molecular level, most of these conventional radioprotectants exert their effects primarily by scavenging free radicals and ROS induced by IR and acting as hydrogen or electron donors to assist cellular repair of damaged macromolecules (Giambarresi and Jacobs, 1987; Poggi et al., 2001). They also can bind directly to certain cellular macromolecules and facilitate repair of the damaged molecules or form mixed disulfide bonds with thiol proteins and alter intrinsic radiosensitivity (Giambarresi and Jacobs, 1987; Poggi et al., 2001). At the physiological level, some of the radioprotectants can cause hypotension/hypoxia/hypothermia to reduce IR-induced oxidative damage or induce Phase II detoxification enzymes to facilitate tissue repair (Giambarresi and Jacobs, 1987; Poggi et al., 2001). In general, these conventional radioprotectants can only be used as prophylactic agents (Giambarresi and Jacobs, 1987). They must be administered prior to exposure to IR in order to achieve an effective protection. This is because within 10⁻⁷ and 10⁻³ seconds after exposure to IR, the reactions of most IR-generated free radicals/ROS with target macromolecules are essentially complete. Moreover, the oxidative damage becomes fixed within a few seconds or minutes and cannot readily be reversed via simple donation of hydrogen or electrons by radioprotectants (Giambarresi and Jacobs, 1987). The repair of these fixed damages, if it occurs at all, requires a much slower process wherein endogenous enzymes remove the reaction products and repair the chemical lesions produced in damaged cell, provided that they can survive long enough.

In addition, certain cytokines have been shown to function as radioprotectants (Neta and Okunieff, 1996). Some of these, such as the interleukins (IL-1-IL-8) and tumor necrosis factor, act to induce production of endogenous antioxidant enzymes and thus, require pre-administration to mediate radiation protection. Others, including certain growth factors for various types of cells, promote tissue repair and recovery by inhibiting IR-induced cell death and stimulating cell proliferation. These growth factors also represent a new frontier of research for radiation protection but will not be studied in this application.

VI. Combination Treatments

In order to increase the effectiveness of an anti-radiation therapy, it may be desirable to combine caspase inhibitors with other agents effective in the treatment or prevention of radiation injury. Such other agents include conventional radioprotectants and anti-apoptotic molecules. The radioprotectant may be a second caspase inhibitor, an anti-apoptotic molecule (a p53 inhibitor or an anti-apoptotic protein, such as Bcl-X_{L}, Bcl-2, c-IAP1, c-IAP2, and XIAP), a radioprotectant (amifostine, vitamin E, vitamin C, selenium, melatonin, 5-androstenediol, cucumin, α-phenyl-tert-butylnitrone, a flavinoid, or a nitroxide), a cytokine (IL-1, IL-2, IL-3, IL -4, IL-5, IL-6, IL-7), or a growth factor (Flt3 ligand, c-Kit ligand, M-CSF, GM-CSF, G-CSF, VEGF, erythropoietin, leukemia inhibitory factor). The inhibitor of apoptosis may be an anti-apoptotic protein, such as Bcl-X_{L}, Bcl-2, apoptosis family proteins (c-IAP1, c-LAP2, and XIAP).

These compositions will be provided in a combined amount effective to protect or treat a cell/tissue/subject from IR damage. This process may involve contacting the cell/tissue/subject with the agents at the same time. This may be achieved by contacting the cell/tissue/subject with a single composition or pharmacological formulation that includes both agents, or by contacting the cell/tissue/subject with two distinct compositions or formulations at the same time, wherein one composition includes the caspase inhibitor and the other includes the second agent. The administration of one or both agents may also be “continuous,” effected by virtue of timed-release delivery vehicles, or continuous infusion/perfiision.

Alternatively, one composition may precede or follow the other agent treatment by various intervals. In certain embodiments, the caspase inhibitor will be reserved for post-radiation treatment, whereas the conventional radioprotectant will be administered on an ongoing basis where the subject is at risk of radiation exposure. Various combinations may be employed, as shown below, where caspase inhibitor therapy is “A” and the secondary agent is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the agents of the present invention to a patient will follow general protocols for the administration of pharmaceuticals, taking into account the toxicity, if any, of the agent. It is expected that the treatment cycles would continue or be repeated as necessary.

VII. Methods of Screening

The present invention further comprises methods for identifying caspase inhibitors that are capable of inhibiting apoptosis in bone marrow cells, in particular, in hematopoietic stem cells. Such assays may comprise random screening of large libraries of candidate substances for caspase inhibition, or the assays may focus on particular known caspase inhibitors selected with an eye towards their ability to function particularly as inhibitors of IR-induced apoptosis.

To identify a suitable inhibitor, one generally will determine the ability of the inhibitor to abrogate or limit injury induced by ionizing radiation. For example, a method generally comprises:
(a) providing a candidate inhibitor;
(b) subjecting an HSC cell to a dose of radiation sufficient to induce radiation injury;
(c) measuring one or more characteristics of radiation injury in the cell; and
(d) comparing the characteristic measured in step (c) with the same characteristic in a cell not treated with the candidate inhibitor,
   wherein a difference between the measured characteristics indicates that said candidate inhibitor is, indeed, an inhibitor of radiation-induced injury.

Assays may be conducted in isolated cells, tissues, or in whole organisms. It will, of course, be understood that all the screening methods of the present invention are useful in themselves notwithstanding the fact that effective inhibitors may not be found. The invention provides methods for screening for such candidates, not solely methods of finding them.

1. Modulators

As used herein the term “candidate substance” refers to any molecule that may potentially inhibits caspase activity and radiation injury. The candidate substance may be a protein or fragment thereof, a small molecule, or even a nucleic acid molecule. It may prove to be the case that the most useful pharmacological compounds will be compounds that are structurally related to existing caspase inhibitors. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one which exerts its inhibitory effect upstream, downstream or directly on caspases. Regardless of the type of inhibitor or activator identified by the present screening methods, the effect of the inhibition or activator by such a compound results in improved response to radiation injury as compared to that observed in the absence of the candidate.

2. In Vitro Assays

A quick, inexpensive and easy assay to run is an in vitro assay. Such assays generally use cells, can be run quickly and in large numbers, thereby increasing the amount of information obtainable in a short period of time. A variety of vessels may be used to run the assays, including test tubes, plates, dishes and other surfaces such as dipsticks or beads.

3. In Vivo Assays

The present invention also contemplates the screening of compounds for their ability to inhibit caspases in various animal models. Due to their size, ease of handling, and information on their physiology and genetic make-up, mice are a preferred model. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for inhibitors may be conducted using an animal model derived from any of these species.

In such assays, one or more candidates are administered to an animal, either before or after radiation insult, and the ability of the candidate to alter one or more characteristics of radiation insult, such as characteristics of apoptosis, as compared to a similar animal not treated with the candidate, identifies an inhibitor. The characteristics may Annexin-V staining, caspase activation, and/or DNA fragmentation, although others are appropriate as well.

The present invention provides methods of screening candidate inhibitors- in combination with other agents, such as conventional radioprotectants. This method will follow the general outline set forth above, including the steps of administering both candidates to the animal and determining the relevant characteristics at appropriate time points. Controls may involve treatment with no candidate and/or either candidate alone. Also, measuring toxicity and dose response can be performed.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

4. Assays for Apoptosis

In accordance with the present invention, one will want to assess various of the characteristics of apoptosis. Typically, the characteristics of apoptosis are Annexin-V staining, caspase activation, and DNA fragmentation. Thus, any assay that examines one or more of these characteristics may be employed.

For example, one may look at DNA fragmentation using a separative method, e.g., chromatography or electrophoresis, to size fractionate the sample. An exemplary assay involves the isolation of DNA from cells, followed by agarose gel electrophoresis and staining with ethidium bromide. DNA fragmentation, characteristic of apoptosis, will be visualized as “ladders” containing a wide range of fragment sizes.

One may employ terminal deoxynucleotidyl transferase mediated dUTP-biotin nick end labeling (TUNEL) assays to measure the integrity of DNA (Gorczyca, 1993). This assay measures the fragmentation of DNA by monitoring the incorporation of labeled UTP into broken DNA strands by the enzyme terminal transferase. The incorporation can be monitored by electroscopy or by cell sorting methodologies (e.g., FACS).

One also may examine cells using standard light or electron microscopy to assess the presence or absence of the cytopatliologies characteristic of apoptosis. Those of skill in the art, applying standard methods of microscopy, will be able to assess cytopathology. In a variation, one may use microscopy in combination with staining procedures, such as Annexin V-7AAD or PI staining. Also contemplated is sub G0/1 cell analysis.

Finally, though an indirect assessment of apoptosis, one may employ caspase activity assays. Commercial kits are available, for example, from Chemicon International (CleavaLite™ Bioluminescent Caspase-3 Activity Assay Kit) and Roche Diagnostics (Caspase 3 Activity Assay).

VIII. DNA Vectors

DNA vectors form important further aspects of the present invention. The use of these vectors to express proteins. The term “expression vector or construct” means any type of genetic construct containing a nucleic acid coding for a gene product in which part or all of the nucleic acid encoding sequence is capable of being transcribed into mRNA. The transcript may be translated into a protein, but it need not be. Thus, in certain embodiments, expression includes both transcription of a gene and the translation of its RNA into a gene product (protein). In other embodiments, expression only includes transcription of the nucleic acid, for example, to generate antisense constructs.

Particularly useful vectors are contemplated to be those vectors in which the coding portion of the DNA segment, whether encoding a full length protein or smaller peptide, is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned”, “under control” or “under transcriptional control” means that the promoter is in the correct location and orientation in relation to the nucleic acid to control RNA polymerase initiation and expression of the gene.

The promoter may be in the form of the promoter that is naturally associated with a gene encoding a bone cell spheroid enhancing protein, as may be obtained by isolating the 5′ non-coding sequences located upstream of the coding segment or exon, for example, using recombinant cloning and/or PCR technology, in connection with the compositions disclosed herein.

Naturally, it will be important to employ a promoter that effectively directs the expression of the DNA segment in the cell type, organism, or even animal, chosen for expression. The use of promoter and cell type combinations for protein expression is generally known to those of skill in the art of molecular biology, for example, see Sambrook et al. (2001), incorporated herein by reference. The promoters employed may be constitutive, or inducible, and can be used under the appropriate conditions to direct high level expression of the introduced DNA segment, such as is advantageous in the large-scale production of recombinant proteins or peptides.

Of particular use are promoters and enhancers that direct transcription of genes that are specific for or highly expressed in bone tissue, osteoblasts and bone precursor cells. For instance, the promoter and enhancer elements of type I collagen, alkaline phosphatase, other bone matrix proteins such as osteopontin, osteonectin and osteocalcin, as well as c-Fos, which is expressed in large amounts in bone and cartilaginous tissues in the generation process, would all be useful for the expression of bone cell spheroid enhancing constructs of the present invention.

In various other embodiments, the human cytomegalovirus (CMV) immediate early gene promoter, the SV40 early promoter and the Rous sarcoma virus long terminal repeat can be used to obtain high-level expression of nucleic acids. The use of other viral or mammalian cellular or bacterial phage promoters which are well-known in the art to achieve expression are contemplated as well, provided that the levels of expression are sufficient for a given purpose.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

Additionally any promoter/enhancer combination (as per the Eukaryotic Promoter Data Base EPDB) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Both cDNA and genomic sequences are suitable for eukaryotic expression, as the host cell will generally process the genomic transcripts to yield functional mRNA for translation into protein. Generally speaking, it may be more convenient to employ as the recombinant gene a cDNA version of the gene. It is believed that the use of a cDNA version will provide advantages in that the size of the gene will generally be much smaller and more readily employed to transfect the targeted cell than will a genomic gene, which will typically be up to an order of magnitude or more larger than the cDNA gene. However, it is contemplated that a genomic version of a particular gene may be employed where desired.

The term “antisense nucleic acid” is intended to refer to the oligonucleotides complementary to the base sequences of DNA and RNA. Antisense oligonucleotides, when introduced into a target cell, specifically bind to their target nucleic acid and interfere with transcription, RNA processing, transport and/or translation. Targeting double-stranded (ds) DNA with oligonucleotide leads to triple-helix formation; targeting RNA will lead to double-helix formation.

Antisense constructs may be designed to bind to the promoter and other control regions, exons, introns or even exon-intron boundaries of a gene. Antisense RNA constructs, or DNA encoding such antisense RNAs, may be employed to inhibit gene transcription or translation or both within a host cell, either in vitro or in vivo, such as within a host animal, including a human subject. Nucleic acid sequences comprising “complementary nucleotides” are those which are capable of base-pairing according to the standard Watson-Crick complementary rules. That is, that the larger purines will base pair with the smaller pyrimidines to form only combinations of guanine paired with cytosine (G:C) and adenine paired with either thymine (A:T), in the case of DNA, or adenine paired with uracil (A:U) in the case of RNA.

As an alternative to targeted antisense delivery, targeted ribozymes may be used. The term “ribozyme” refers to an RNA-based enzyme capable of targeting and cleaving particular base sequences in oncogene DNA and RNA. Ribozymes either can be targeted directly to cells, in the form of RNA oligo-nucleotides incorporating ribozyme sequences, or introduced into the cell as an expression construct encoding the desired ribozymal RNA. Ribozymes may be used and applied in much the same way as described for antisense nucleic acids.

It is proposed that proteins, polypeptides or peptides may be co-expressed with other selected proteins, wherein the proteins may be co-expressed in the same cell or a gene may be provided to a cell that already has another. selected protein. Co-expression may be achieved by co-transfecting the cell with two distinct recombinant vectors, each bearing a copy of either of the respective DNA. Alternatively, a single recombinant vector may be constructed to include the coding regions for both of the proteins, which could then be expressed in cells transfected with the single vector. In either event, the term “co-expression” herein refers to the expression of both a bone cell spheroid enhancing gene and the other selected protein in the same recombinant cell.

IX. Methods of Gene Transfer

In order to mediate the effect transgene expression in a cell, it will be necessary to transfer the expression vectors of the present invention into a cell. Such transfer may employ viral or non-viral methods of gene transfer. This section provides a discussion of methods and compositions of gene transfer.

A. Viral Vector-Mediated Transfer

The bone spheroid enhancing constructs may be incorporated into an infectious particle to mediate gene transfer to a cell. Additional expression constructs as described herein may also be transferred via viral transduction using infectious viral particles, for example, by transformation with an adenovirus vector of the present invention as described herein below. Alternatively, lentiviral, retroviral or bovine papilloma virus may be employed, both of which permit permanent transformation of a host cell with a gene(s) of interest. Thus, in one example, viral infection of cells is used in order to deliver therapeutically significant genes to a cell. Typically, the virus simply will be exposed to the appropriate host cell under physiologic conditions, permitting uptake of the virus. Though adenovirus is exemplified, the present methods may be advantageously employed with other viral vectors, as discussed below.

Adenovirus. Adenovirus is particularly suitable for use as a gene transfer vector because of its mid-sized DNA genome, ease of manipulation, high titer, wide target-cell range, and high infectivity. The roughly 36 kB viral genome is bounded by 100-200 base pair (bp) inverted terminal repeats (ITR), in which are contained cis-acting elements necessary for viral DNA replication and packaging. The early (E) and late (L) regions of the genome that contain different transcription units are divided by the onset of viral DNA replication.

The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression, and host cell shut off (Renan, 1990). The products of the late genes (L1, L2, L3, L4 and L5), including the majority of the viral capsid proteins, are expressed only after significant processing of a single primary transcript issued by the major late promoter (MLP). The MLP (located at 16.8 map units) is particularly efficient during the late phase of infection, and all the mRNAs issued from this promoter possess a 5′ tripartite leader (TL) sequence which makes them preferred mRNAs for translation.

In order for adenovirus to be optimized for gene therapy, it is necessary to maximize the carrying capacity so that large segments of DNA can be included. It also is very desirable to reduce the toxicity and immunologic reaction associated with certain adenoviral products. The two goals are, to an extent, coterminous in that elimination of adenoviral genes serves both ends. By practice of the present invention, it is possible achieve both these goals while retaining the ability to manipulate the therapeutic constructs with relative ease.

The large displacement of DNA is possible because the cis elements required for viral DNA replication all are localized in the inverted terminal repeats (ITR) (100-200 bp) at either end of the linear viral genome. Plasmids containing ITR's can replicate in the presence of a non-defective adenovirus (Hay et al., 1984). Therefore, inclusion of these elements in an adenoviral vector should permit replication.

In addition, the packaging signal for viral encapsidation is localized between 194-385 bp (0.5-1.1 map units) at the left end of the viral genome (Hearing et al., 1987). This signal mimics the protein recognition site in bacteriophage λ DNA where a specific sequence close to the left end, but outside the cohesive end sequence, mediates the binding to proteins that are required for insertion of the DNA into the head structure. E1 substitution vectors of Ad have demonstrated that a 450 bp (0-1.25 map units) fragment at the left end of the viral genome could direct packaging in 293 cells (Levrero et al., 1991).

Previously, it has been shown that certain regions of the adenoviral genome can be incorporated into the genome of mammalian cells and the genes encoded thereby expressed. These cell lines are capable of supporting the replication of an adenoviral vector that is deficient in the adenoviral function encoded by the cell line. There also have been reports of complementation of replication deficient adenoviral vectors by “helping” vectors, e.g., wild-type virus or conditionally defective mutants.

Replication-deficient adenoviral vectors can be complemented, in trans, by helper virus. This observation alone does not permit isolation of the replication-deficient vectors, however, since the presence of helper virus, needed to provide replicative functions, would contaminate any preparation. Thus, an additional element was needed that would add specificity to the replication and/or packaging of the replication-deficient vector. That element, as provided for in the present invention, derives from the packaging function of adenovirus.

It has been shown that a packaging signal for adenovirus exists in the left end of the conventional adenovirus map (Tibbetts, 1977). Later studies showed that a mutant with a deletion in the E1A (194-358 bp) region of the genome grew poorly even in a cell line that complemented the early (E1A) function (Hearing and Shenk, 1983). When a compensating adenoviral DNA (0-353 bp) was recombined into the right end of the mutant, the virus was packaged normally. Further mutational analysis identified a short, repeated, position-dependent element in the left end of the Ad5 genome. One copy of the repeat was found to be sufficient for efficient packaging if present at either end of the genome, but not when moved towards the interior of the Ad5 DNA molecule (Hearing et al., 1987).

By using mutated versions of the packaging signal, it is possible to create helper viruses that are packaged with varying efficiencies. Typically, the mutations are point mutations or deletions. When helper viruses with low efficiency packaging are grown in helper cells, the virus is packaged, albeit at reduced rates compared to wild-type virus, thereby permitting propagation of the helper. When these helper viruses are grown in cells along with virus that contains wild-type packaging signals, however, the wild-type packaging signals are recognized preferentially over the mutated versions. Given a limiting amount of packaging factor, the virus containing the wild-type signals are packaged selectively when compared to the helpers. If the preference is great enough, stocks approaching homogeneity should be achieved.

Retrovirus. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes—gag, pol and env—that code for capsid proteins, polymerase enzyme, and envelope components, respectively. A sequence found upstream from the gag gene, termed Ψ, functions as a signal for packaging of the genome into virions. Two long terminal repeat (LTR) sequences are present at the 5′ and 3′ ends of the viral genome. These contain strong promoter and enhancer sequences and also are required for integration in the host cell genome (Coffin, 1990).

In order to construct a retroviral vector, a nucleic acid encoding a promoter is inserted into the viral genome in the place of certain viral sequences to produce a virus that is replication-defective. In order to produce virions, a packaging cell line containing the gag, pol and env genes but without the LTR and Ψ components is constructed (Mann et al., 1983). When a recombinant plasmid containing a human cDNA, together with the retroviral LTR and Ψ sequences is introduced into this cell line (by calcium phosphate precipitation for example), the Ψ sequence allows the RNA transcript of the recombinant plasmid to be packaged into viral particles, which are then secreted into the culture media (Nicolas and Rubenstein, 1988; Temin, 1986; Mann et aL, 1983). The media containing the recombinant retroviruses is collected, optionally concentrated, and used for gene transfer. Retroviral vectors are able to infect a broad variety of cell types. However, integration and stable expression of many types of retroviruses require the division of host cells (Paskind et al., 1975).

An approach designed to allow specific targeting of retrovirus vectors recently was developed based on the chemical modification of a retrovirus by the chemical addition of galactose residues to the viral envelope. This modification could permit the specific infection of cells such as hepatocytes via asialoglycoprotein receptors, should this be desired.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, the infection of a variety of human cells that bore those surface antigens was demonstrated with an ecotropic virus in vitro (Roux et al., 1989).

Adeno-associated Virus. AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription.

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

The terminal repeats of the AAV vector can be obtained by restriction endonuclease digestion of AAV or a plasmid such as p201, which contains a modified AAV genome (Samulski et aL 1987), or by other methods known to the skilled artisan, including but not limited to chemical or enzymatic synthesis of the terminal repeats based upon the published sequence of AAV. The ordinarily skilled artisan can determine, by well-known methods such as deletion analysis, the minimum sequence or part of the AAV ITRs which is required to allow function, i.e., stable and site-specific integration. The ordinarily skilled artisan also can determine which minor modifications of the sequence can be tolerated while maintaining the ability of the terminal repeats to direct stable, site-specific integration.

AAV-based vectors have proven to be safe and effective vehicles for gene delivery in vitro, and these vectors are being developed and tested in pre-clinical and clinical stages for a wide range of applications in potential gene therapy, both ex vivo and in vivo (Carter and Flotte, 1995; Chatterjee et al., 1995; Ferrari et al., 1996; Fisher et al., 1996; Flotte et al., 1993; Goodman et al., 1994; Kaplitt et al., 1994; 1996, Kessler et al., 1996; Koeberl et al., 1997; Mizukami et al., 1996; Xiao et al., 1996).

AAV-mediated efficient gene transfer and expression in the lung has led to clinical trials for the treatment of cystic fibrosis (Carter and Flotte, 1995; Flotte et al., 1993). Similarly, the prospects for treatment of muscular dystrophy by AAV-mediated gene delivery of the dystrophin gene to skeletal muscle, of Parkinson's disease by tyrosine hydroxylase gene delivery to the brain, of hemophilia B by Factor IX gene delivery to the liver, and potentially of myocardial infarction by vascular endothelial growth factor gene to the heart, appear promising since AAV-mediated transgene expression in these organs has recently been shown to be highly efficient (Fisher et al., 1996; Flotte et al., 1993; Kaplitt et al., 1994; 1996; Koeberl et al., 1997; McCown et al., 1996; Ping et al., 1996; Xiao et al., 1996).

Lentivirus. Lentivirus vectors based on human immunodeficiency virus (HIV) type 1 (HIV-1) constitute a recent development in the field of gene therapy. A key property of HIV-1-derived vectors is their ability to infect nondividing cells. High-titer HIV- 1-derived vectors have been produced. Examples of lentiviral vectors include White et al. (1999), describing a lentivirus vector which is based on HIV, simian immunodeficiency virus (SIV), and vesicular stomatitis virus (VSV) and which the inventors refer to as HIV/SIVpack/G. The potential for pathogenicity with this vector system is minimal. The transduction ability of HIV/SIVpack/G was demonstrated with immortalized human lymphocytes, human primary macrophages, human bone marrow-derived CD34(+) cells, and primary mouse neurons. Gasmi et al. (1999) describe a system to transiently produce HIV-1-based vectors by using expression plasmids encoding gag, pol, and tat of HIV-1 under the control of the cytomegalovirus immediate-early promoter.

Other Viral Vectors. Other viral vectors may be employed as expression constructs in the present invention. Vectors derived from viruses such as vaccinia virus (Ridgeway, 1988; Baichwal and Sugden, 1986; Coupar et al., 1988) canary pox virus, and herpes viruses may be employed. These viruses offer several features for use in gene transfer into various mammalian cells.

B. Non-viral Transfer

DNA constructs of the present invention are generally delivered to a cell, in certain situations, the nucleic acid to be transferred is non-infectious, and can be transferred using non-viral methods.

Several non-viral methods for the transfer of expression constructs into cultured mammalian cells are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), and receptor-mediated transfection (Wu and Wu, 1987; Wu and Wu, 1988).

Once the construct has been delivered into the cell the nucleic acid encoding the bone cell spheroid enhancing construct may be positioned and expressed at different sites. In certain embodiments, the nucleic acid encoding the therapeutic gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In a particular embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). The addition of DNA to cationic liposomes causes a topological transition from liposomes to optically birefringent liquid-crystalline condensed globules (Radler et al., 1997). These DNA-lipid complexes are potential non-viral vectors for use in gene therapy.

Liposome-mediated nucleic acid delivery and expression of foreign DNA in vitro has been very successful. Using the β-lactamase gene, Wong et al. (1980) demonstrated the feasibility of liposome-mediated delivery and expression of foreign DNA in cultured chick embryo, HeLa, and hepatoma cells. Nicolau et al. (1987) accomplished successful liposome-mediated gene transfer in rats after intravenous injection. Also included are various commercial approaches involving “lipofection” technology.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear nonhistone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention.

Other vector delivery systems which can be employed to deliver a nucleic acid encoding a therapeutic gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferring (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0 273 085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a therapeutic gene also may be specifically delivered into a cell type such as prostate, epithelial or tumor cells, by any number of receptor-ligand systems with or without liposomes. For example, the human prostate-specific antigen (Watt et al., 1986) may be used as the receptor for mediated delivery of a nucleic acid in prostate tissue.

In another embodiment of the invention, the expression construct may simply consist of naked recombinant DNA or plasmids. Transfer of the construct may be performed by any of the methods mentioned above which physically or chemically permeabilize the cell membrane. This is applicable particularly for transfer in vitro, however, it may be applied for in vivo use as well. Dubensky et al. (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. It is envisioned that DNA encoding a CAM may also be transferred in a similar manner in vivo and express CAM.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

### X. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Reagents. PE-conjugated anti-Sca-1 (Clone E13-161.7; rat IgG2a), APC-conjugated anti-c-kit (Clone 2B8; rat IgG2b), biotin-conjugated anti-CD5 (Clone 53-7.3, rat IgG2a), anti-CD45R/B220 (Clone RA3-6B2; rat IgG2a), anti-Gr-1 (Clone RB6-8C5; rat IgG2b), anti-Mac-1 (Clone M1/70; rat IgG2b) and anti-Ter-119 (Clone Ter-119, rat IgG2b), purified rat anti-CD16/CD32 (Clone 2.4G2, Fcγ receptor blocker, rat IgG2b), and FITC-conjugated streptavidin were purchased from Pharmingen (San Diego, Calif.). Z-Val-Ala-Asp (OCH3)-Fluoromethylketone, methyl ester (z-VAD) was purchased from Biomol (Plymouth Meeting, Pa.).

Mice. Male C57BL/6 mice were purchased from The Jackson Laboratories (Bar Harbor, Me.) and housed 4 to a cage at the Medical University of South Carolina AAALAC certified animal facility. They received food and water ad libitum. All mice were used at approximately 8-10 weeks of age. The Institutional Animal Care and Use Committee of the Medical University of South Carolina approved all experimental procedures used in this study.

Isolation of lineage negative hematopoietic (Lin) cells: BM mononuclear cells were incubated with biotin-conjugated rat antibodies specific for murine CD5, Mac-1, CD45R/B220, Ter-119 and Gr-1. The labeled mature lymphoid and myeloid cells were removed twice after they were incubated with goat anti-rat IgG paramagnetic beads (Dynal Inc, Lake Success, N.Y.) at a bead:cell ratio of approximately 4:1 and exposed to a magnetic field. The isolated Lin- cells were washed twice with 2% FCS/HBSS and resuspended in complete medium (RPMI1640 medium with 10%FCS, 200 mM L-glutamine and 100 U/ml penicillin and streptomycin) at 1×10⁶/ml.

Ionizing radiation (IR). Aliquots of 2 ml of Lin- cells (1×10⁶/ml in complete medium) were placed into a cell culture tube and exposed to 4 Gy IR on a rotating platform in a Mark IV¹³⁷Cesium gamma-irradiator (JL Shepherd, Glendale, Calif.) at a dose rate of 1.21 Gy/min.

Apoptosis assays. Lin^{- cells (}1×10⁶/ml in complete medium) were exposed to 4 Gy IR or non-irradiated (Control). They were incubated in wells of a 24-well plate at 37° C., 5% CO₂ and 100% humidity for 6 or 18 h. For z-VAD treatment studies, the cells were pre-incubated with vehicle (0.2% DMSO) or 100 μM z-VAD for 1 h prior to IR exposure, or they were treated with z-VAD immediately before or 30 min, 1 h, 2 h or 4 h after IR exposure. After 6 or 18 h incubation, 0.5 ml cells (5×105) were washed once with 1 ml 0.1% BSA/PBS. The cells were centrifuged for 5 min, 350× g at 4° C. Next, the cells were incubated with anti-CD16/32 at 4° C. for 15 min to block the Fcγ receptors and then stained with Sca-1-PE and c-kit-APC antibodies for 20 min at 4° C. in the dark. The cells were washed twice with 1 ml 0.1% BSA/PBS, centrifuged for 5 min, 350× g at RT. Next, 100 μl 1× binding buffer (10 mM HEPES, pH 7.4, 140 mM NaCl, and 2.5 mM CaCl₂) was added to the pellet along with 3 μl Annexin V-FITC (Pharmingen, San Diego, Calif.) and 5 μl 7-AAD (10 μg/ml, from Molecular Probes, Eugene, Oreg.). The cell suspension was gently mixed and incubated for 15 min at room temperature. The cells were diluted in 400 μl 1× binding buffer immediately prior to subjection of FACS analysis. In all experiments, PE and APC isotype controls and FITC and 7-AAD positive controls were included as appropriate. Two or four-color flow cytometric analyses were performed using a FACS Caliber (Becton Dickinson, San Jose, Calif.).

Statistical Analysis. The data were analyzed by analysis of variance. In the event that analysis of variance justifies post hoc comparisons between group means, these were conducted using the Student-Newman-Keuls test for multiple comparisons. For experiments in which only single experimental and control groups were used, group differences were examined by unpaired Student's t test. Differences were considered significant at p<0.05.

### Example 2

### Results

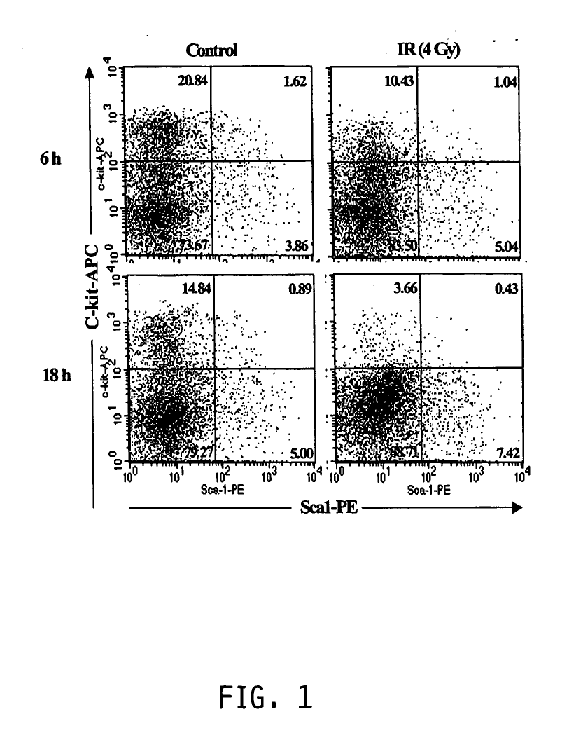

Exposure of Lin⁻ cells to IR selectively decreases Lin⁻ Sca1⁺ c-kit⁺ and Lin⁻ Sca1⁺ c-kit⁺ cells. To examine the effect of IR on different populations of hematopoietic cells, Lin⁻ cells were exposed to 4 Gy IR and analyzed by flow cytometry after immunostaining with the antibodies specifically against Scal and c-kit antigens. A representative flow cytometric analysis of the cells was shown in FIG. 1 and the summary of the analysis was presented in Table 1. As shown in FIG. 1 and Table 1, IR exposure significantly reduced the percentage of Lin⁻ Sca1⁺ c-kit⁺ and Lin⁻ Sca1⁻ c-kit⁺ cells but increased that of Lin⁻ Sca1⁺ c-kit⁻ and Lin⁻ Sca1⁻ c-kit⁻ cells in a time-dependent manner (p<0.001). Since a decrease in the percentage of one cell population in Lin⁻ cells may result in a reciprocal increase in another, the changes in the absolute numbers of each of these cell populations were calculated and presented in Table 2. The results showed that IR exposure significantly reduced the numbers of Lin⁻ Sca1⁺ c-kit⁻, Lin⁻ Sca1⁻ c-kit⁺ and Lin⁻ Sca1⁻ c-kit⁻ cells (p<0.001), but had minimal effect on that of Lin⁻ Sca1⁺ c-kit⁻ cells (p>0.05). The reduction of Lin⁻ Sca1⁺ c-kit⁺ cells was the greatest (64% and 85% reduction at 6 and 18 h, respectively), which was followed by that of Lin⁻ Sca1⁺ c-kit⁺ cells (55% and 65% reduction at 6 and 18 h, respectively), while the reduction of Lin⁻ Sca1⁻ c-kit⁻ cells was moderate (22% and 26% reduction at 6 and 18 h, respectively). It has been shown that Lin⁻ Sca1⁺ c-kit⁻ cell represent enriched HSC that have the ability to give rise to long term multilineage reconstitution (Okada et al., 1992). Lin⁻ Sca1⁻ c-kit⁺ cells are progenitors that are only capable of short term hematopoietic reconstitution (Okada et al., 1992). Conversely, Lin⁻ Sca1⁺ c-kit⁻ and Lin⁻ Sca1⁻ c-kit⁻ cells are devoid of both HSC and progenitors (Okada et al., 1992). Thus, these results suggest that exposure of Lin⁻ cells to IR selectively decreases the numbers of HSC and progenitors.

TABLE 1
Phenotypic Changes of Lin⁻ Cells after Exposure to IR

| | Lin⁻ Sca-1⁺ c-kit⁺ | Lin⁻ Sca-1⁺ c-kit⁺ | Lin⁻ Sca-1⁺ c-kit⁻ | Lin⁻ Sca-1⁻ c-kit⁻ |
|---|---|---|---|---|
| Treatment | (×10⁵) | (×10⁵) | (×10⁵) | (×10⁵) |
| Control (6 h) | 0.133 ± 0.001 | 1.781 ± 0.065 | 0.323 ± 0.015 | 6.096 ± 0.177 |
| IR (6 h) | 1.06 ± 0.02* | 10.45 ± 0.41* | 5.07 ± 0.09* | 83.29 ± 0.34* |
| Control (18 h) | 0.86 ± 0.03 | 15.34 ± 0.50 | 4.66 ± 0.29 | 79.14 ± 0.21 |
| IR (18 h) | 0.46 ± 0.05* | 3.49 ± 0.14* | 7.69 ± 0.52* | 88.35 ± 0.70* |

Lin⁻ cells (1 × 10⁶/ml) were non-irradiated (Control) or exposed to 4 Gy IR. After 6 or 18 h incubation, the cells were stained with Sca-1-PE and c-kit-APC antibodies and analyzed by flow cytometry (a minimal of 150,000 events/sample). The percentage of each phenotype of Lin⁻ cells is presented as mean ± SD of triplicates. Similar results were observed in two additional independent experiments.
*p < 0.001 vs Control.

TABLE 2
Changes in the Numbers of Different Phenotypic Lin⁻ Cells after Exposure to IR

| | Lin⁻ Sca-1⁺ c-kit⁺ | Lin⁻ Sca-1⁻ c-kit⁺ | Lin⁻ Sca-1⁺ c-kit⁻ | Lin⁻ Sca-1⁻ c-kit⁻ |
|---|---|---|---|---|
| Treatment | (%) | (%) | (%) | (%) |
| Control (6 h) | 1.59 ± 0.05 | 21.37 ± 0.57 | 3.88 ± 0.08 | 73.15 ± 0.54 |
| IR (6 h) | 0.061 ± 0.003* | 0.599 ± 0.013* | 0.291 ± 0.009 | 4.776 ± 0.203* |
| Control (18 h) | 0.058 ± 0.002 | 1.034 ± 0.073 | 0.313 ± 0.009 | 5.329 ± 0.239 |
| IR (18 h) | 0.021 ± 0.003* | 0.156 ± 0.01* | 0.344 ± 0.032 | 3.946 ± 0.070* |
| Lin⁻ cells (1 × 10⁶/ml) were non-irradiated (Control) or exposed to 4 Gy IR. After 6 or 18 h incubation, the cells were harvested and counted and then were stained with Sca-1-PE and c-kit-APC antibodies and analyzed by flow cytometry (a minimal of 150,000 events/sample). The numbers of each phenotype of Lin⁻ cells were calculated by multiplication of the total numbers of cells harvested with the percentage | | | | |
| # of each phenotype of Lin⁻ cells determined by flow cytometric analysis. The data are presented as mean ± SD of triplicates. Similar results were observed in two additional independent experiments. | | | | |

*p < 0.001 vs Control.

IR induces apoptosis in Lin⁻ Sca1⁺ c-kit⁺ and Lin⁻ Sca1⁻ c-kit⁻ cells. To determine if IR decreases HSC and progenitors by induction of apoptosis and/or necrosis, Lin⁻ Sca1⁺ c-kit⁺ and Lin⁺ Sca1⁻ c-kit⁺ cells were gated as defined in FIG. 1 and reanalyzed for apoptosis and/or necrosis after counter staining with 7-AAD and annexin-V-FITC. The early and late stage apoptotic cells are stained with annexin V (annexin V⁺) and annexin V plus 7-AAD (annexin V⁺/7-AAD⁺), respectively (Hasper et al., 2000). The necrotic cells are stained with 7-AAD (7-AAD⁺) only, whereas the viable cells are double negative (annexin V⁻/7-AAD⁻) (Hasper et al., 2000). A representative flow cytometric analysis of the cells was shown in FIGS. 2A & B. The percentage of annexin V⁺ and annexin V⁺/7-AAD⁺ cells were added together to represent the total numbers of cells undergoing apoptosis (FIGS. 3A & B). As shown in FIGS. 2A & B and 3A & B, at 6 h after IR the percentage of apoptotic cells (annexin V⁺ and annexin V⁺/7-AAD⁺ cells) in Lin⁼ Sca1⁺ c-kit⁺ (IR: 50.43% vs Control: 20.26%) and Lin^{-Sca}1⁻ c-kit⁺ (IR: 31.17% vs Control: 6.75%) cells were significantly increased (p<0.001) and that of necrotic cells (7-AAD⁺ cells) in these cells was unchanged (p>0.05). At 18 h post IR, the percentage of apoptotic cells were further elevated in Lin⁼ Sca1⁺ c-kit⁺ (IR: 64.80% vs° Control: 23.38%, p<0.001) and Lin⁻ Sca1⁻ c-kit⁺ (IR: 46.17% vs Control: 8.25%, p<0.001) cells, while that of necrotic cells in Lin⁻ Sca1⁺ c-kit⁺ (IR: 4.03% vs Control: 1.89%, p<0.001) and Lin⁻ Sca1⁻ c-kit⁺ (IR: 3.25% vs Control: 1.78%, p<0.05) cells was slightly increased. Thus, this result suggests that IR mainly induces cell death in HSC and progenitors by apoptosis.

z-VAD inhibits IR-induced apoptosis and decrease in Lin⁻ Sca1⁺ c-kit⁺ and Lin⁻ Sca1⁻ c-kit⁺ cells. Caspase activation has been implicated in mediating apoptosis induced by many apoptotic stimuli including IR (Earnshaw et al., 1999; Pruschy et al., 2001). z-VAD is a potent and non-specific caspase inhibitor that has the ability to inhibit multiple forms of caspases (Naito et al., 1997; Susin et al., 1999). Inhibition of caspase activities by z-VAD can block apoptosis in a cell type- and stimulus-specific manner (Naito et al., 1997; Susin et al., 1999). To determine if z-VAD is capable of inhibiting IR-induced apoptosis in HSC and progenitors, Lin⁻ cells were pretreated with z-VAD for 1 h and then exposed to IR. The percentage of apoptotic cells in Lin⁻ Sca1⁺ c-kit⁺ and Lin⁻ Sca1⁻ c-kit⁺ cells was analyzed at 6 and 18 h post IR (FIGS. 4A & B and 5A&B). It was found that the pretreatment with z-VAD significantly inhibited IR-induced apoptosis in Lin⁻ Sca1⁺ c-kit⁺ cells at 6 h and completely protected them at 18 h after IR. z-VAD also attenuated IR-induced apoptosis in Lin⁻ Sca1⁻ c-kit⁺ cells, but the effect was less dramatic as compared to that of Lin⁻ Sca1⁺ c-kit⁺ cells. Moreover, the inhibition of IR-induced apoptosis in Lin⁻ Sca1⁺ c-kit⁺ and Lin⁻ Sca1⁻ c-kit⁺ cells by z-VAD did not lead to an increase in necrotic cell death in these cells, suggesting that z-VAD is capable of protecting HSC and progenitors from IR-induced cell death.

Next, the inventors determined if inhibition of apoptosis by z-VAD can prevent HSC and progenitors from IR-induced reduction. The numbers of Lin⁻ Sca1⁺ c-kit⁺ and Lin⁻ Sca1⁻ c-kit⁺ cells were determined at 6 and 18 h after the vehicle- and z-VAD-treated Lin⁻ cells were exposed to IR, and expressed as a percentage of that of un-irradiated control cells. As shown in FIGS. 6A & B, exposure of the vehicle-treated Lin⁻ cells to IR resulted in 40.50% and 69.20% decrease in the numbers of Lin⁻ Sca1⁺ c-kit⁺ cells (p<0.001) and 59.76% and 87.06% decrease in that of Lin⁻ Sca1⁺ c-kit⁺ cells (p<0.001) at 6 and 18 h post IR, respectively. z-VAD pretreatment completely prevented IR-induced reduction of Lin⁻ Sca1⁺ c-kit⁺ cells, while it only partially protected Lin⁻ Sca1⁻ c-kit⁺ cells. Particularly at 18 h post IR, the numbers of Lin⁻ Sca1⁻ c-kit⁺ cells in the z-VAD-treated cells were only about 29% of that in un-irradiated control cells. These findings suggest that the induction of apoptosis by IR is likely the primary cause of IR-induced decrease in HSC, and it also contributes to IR-induced reduction in Lin⁻ Sca1⁻ c-kit⁺ cells. However, other factors, such as down regulation of c-kit expression, may also contribute to IR-induced reduction in Lin⁻ Sca1⁻ c-kit⁺ cells (Randall and Weissman, 1997).

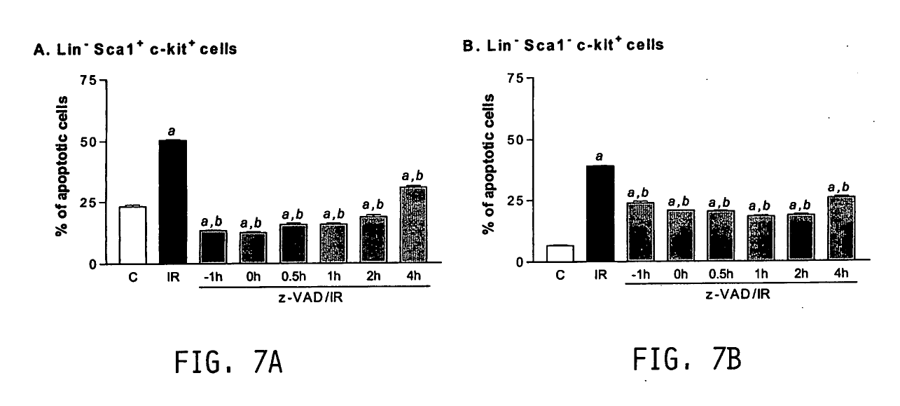

Post IR treatment with z-VAD effectively inhibits IR-induced apoptosis in Lin⁻ Sca1⁺ c-kit⁺ and Lin⁻ Sca1⁻ c-kit⁺ cells. The activation of caspases usually occurs after a cell is stimulated with an apoptotic stimulus (Earnshaw et al., 1999; Pruschy et al., 2001). The time between exposure of a cell to an apoptotic signal to caspase activation may vary depending upon the cell-type and insult (Earnshaw et al., 1999; Pruschy et al., 2001). There may be a time window when post IR treatment with z-AVD is still effective in inhibiting IR-induced apoptosis in HSC and progenitors. To test this hypothesis, Lin⁻ cells were pre-incubated with vehicle (IR) or z-VAD (−1 h) for 1 h prior to IR exposure, or they were treated with z-VAD immediately before (0 h) or 30 min (0.5 h), 1 h, 2 h or 4 h after IR exposure. The percentage of apoptotic cells in these irradiated cells with or without z-VAD treatment was compared to that of un-irradiated control cells (C). As shown in FIGS. 7A & B, the vehicle-treated cells exposed to IR exhibited significant increases in apoptosis in Lin⁻ Sca1⁺ c-kit⁺ (IR: 50.03% vs Control: 23.17%) and Lin⁻ Sca1⁻ c-kit⁺ (IR: 49.61% vs Control: 6.61%) cells. z-VAD treatment prior to IR (-1 h) or up to 2 h post IR abrogated IR-induce apoptosis in Lin⁼ Sca1⁺ c-kit⁺ cells, while the treatment at 4 h post IR only partially inhibited the apoptosis. A similar result was also observed in Lin⁻ Sca1⁺ c-kit⁺ cells, but to a less extent.

z-VAD protects HSC from IR-induced suppression of hematopoietic function. To determine if inhibition of IR-induced HSC apoptosis by z-VAD preserves their hematopoietic function, mouse BM cells were pre-incubated with vehicle (0.2% DMSO) or z-VAD (100 μM) one hour prior to exposure to IR (4 Gy). The hematopoietic function of these cells were analyzed by cobblestone area-forming cell (CAFC) assay (Ploemacher et al., 1989). This in vitro assay provides an estimate of the hematopoietic function of a spectrum of CAFC day-types that correspond to CFU-granulocyte macrophage (CFU-GM) (CAFC day 7), CFU-spleen (CFU-S) day-12 (CAFC day 14), and the primitive HSC with long-term repopulating ability (CAFC day 28-35) (Ploemacher et al., 1989).

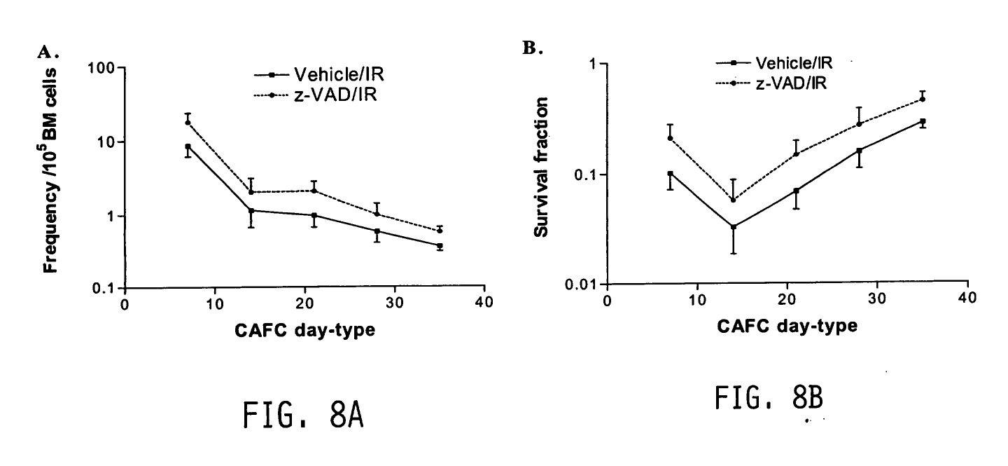

As shown in FIGS. 8A & B, exposure of the vehicle-treated BM cells to IR resulted in significant reduction in the frequency of CAFC day 7-35. CAFC day 14 cells were the most sensitive cell type to IR (survival fraction=0.033), followed by CAFC day 21 (survival fraction=0.697) and day 7 (survival fraction=0.101). All these cells represent different stages of hematopoietic progenitors that are usually very sensitive to IR. CAFC day 28 and 35 were relatively less sensitive to IR and exhibited higher survival fraction than earlier CAFC day-types. CAFC day 28 and day 35 had a survival fraction of 0.157 and 0.288 after exposure to IR, respectively. These findings are similar to that reported previously (Ploemacher et al., 1992). Incubation of BM cells with z-VAD prior to IR exposure significantly increased the CAFC frequency and survival fraction as compared to that of vehicle-treated cells exposed to IR (p<0.01). The survival fractions of CAFC day 7, 14, 21, 28 and 35 were 0.211, 0.058, 0.148, and 0.453, respectively. All these values were about 2-fold greater than those of vehicle-treated cells, indicating that z-VAD not only inhibits IR-induced hematopoietic cell apoptosis but also preserves their hematopoietic function.

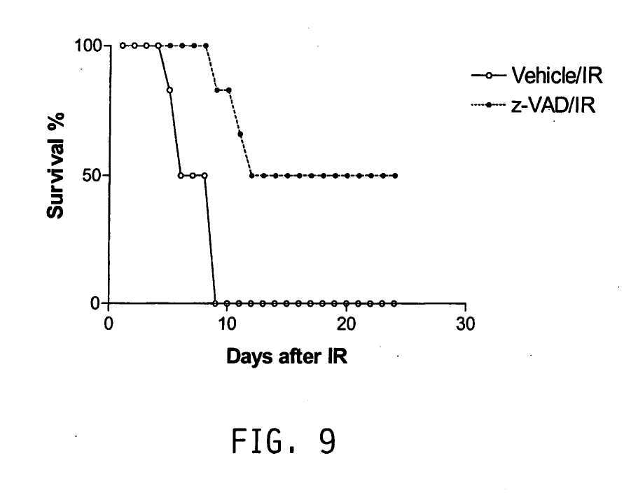

z-VAD protects mice from IR-induced lethality. Finally, the inventors examined if z-VAD acts as a radioprotectant to protect mice from IR-induced death. Male C57BL/6 mice were given two i.p. injections of vehicle (0.25 ml PBS with 0.2% DMSO) or z-VAD (6 mg/kg) at one hour prior to and 5 h after exposure to a lethal dose of IR (10.5 Gy). Exposure of vehicle-treated mice to 10.5 Gy IR resulted in 100% lethality within 9 days (FIG. 9). The death is primarily caused by the oxidative damage of IR to the hematopoietic stem cells in the bone marrow. In contrast, 50% mice receiving z-VAD injections remain alive up to today (24 days) after IR. This demonstrates that z-VAD is a novel radioprotectant that has the ability to protect mice from IR-induced lethality, probably by inhibiting IR-induced HSC apoptosis.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### XI. REFERENCES

The following references, to the extent that they provide exemplary procedural or other details supplementary to those set forth herein, are specifically incorporated herein by reference.
U.S. Pat. No. 6,426,413
U.S. Pat. No. 5,552,400
Baichwal and Sugden, In: Gene Transfer, Kucherlapati (Ed.), NY, Plenum Press, 117-148, 1986.
Benvenisty and Neshif, Proc. Natl. Acad. Sci. USA, 83:9551-9555, 1986.
Carter and Flotte, Gene Ther. 2(6):357-62 1995.
Chatterjee, et al., Ann. NY Acad. Sci., 770:79-90, 1995.
Chen and Okayama, Mol. Cell Biol., 7:2745-2752, 1987.
Chute et al., Mil. Med., 167:74-77, 2002.
Coffin, In: Virology, ed., NY, Raven Press, 1437-1500, 1990.
Coupar et al., Gene, 68:1-10, 1988.
Cui et al., J. Environ. Pathol. Toxicol. Oncol., 14:159-163, 1995.
Devine and Chaput, In: Militory Radiobiology, Conklin and Walker (Eds.), Academic Press, Inc., Orlando, Fla., 380-392, 1987.
Di Leonardo et al., Genes Dev., 8:2540-2551, 1994.
Dolle et al., J. Med. Chem., 39:2438-2440, 1996.
Domen et al., Blood. 91:2272-2282, 1998.
Down et al., Blood, 86:122-127, 1995.
Dubensky et al., Natl. Acad. Sci. USA, 81:7529-7533, 1984.
Earnshaw et al., Annu. Rev. Biochem., 68:383-424, 1999.
EP 273 085
EP 547 699
Estrov et al., Blood, 84:380a, 1994.
Fechheimer et al., Proc. Natl. Acad. Sci. USA, 84:8463-8467, 1987.
Ferkol et al., FASEB J., 7:1081-1091, 1993.
Ferrari et al., J. Virol., 70:3227-3234, 1996.
Fisher et al., J. Virol., 70:520-532, 1996.
Flotte et al., Proc. Natl. Acad. Sci. USA, 90:10613-10617, 1993.
Fraley et al., Proc. Natl. Acad. Sci. USA, 76:3348-3352, 1979.
Gasmi et al., J. Virol., 73(3):1828-34, 1999.
Ghosh and Bachhawat, In: Liver diseases, targeted diagnosis and therapy using specific receptors and ligands, Wu and Wu (Ed.), NY, Marcel Dekker, 87-104, 1991.
Giambarresi and Jacobs, In: Militory Radiobiology, Conklin and Walker (Eds.) Academic Press, Inc., Orlando. 265-301, 1987.
Goodman et al., Blood, 84:1492-1500, 1994.
Gopal, Mol. Cell Biol., 5:1188-1190, 1985.
Gorczyca et al., Cancer Res., 53(8):1945-1951, 1993.
Graham and Van Der Eb, Virology, 52:456-467, 1973.
Granier and Gambini, In: Applied radiobiology and radiation protection, Ellis Horwood, New York, 1990.
Harland and Weintraub, J. Cell Biol., 101:1094-1099, 1985.
Harms-Ringdahl et al., Mutat. Res., 366:171-179, 1996.
Hasper et al., Cytometry, 40:167-171, 2000.
Hay etal., J. Mol. Biol., 175:493-510, 1984.
Hearing and Shenk, J. Mol. Biol., 167:809-822, 1983.
Hearing et al., J. Virol., 67:2555-2558, 1987.
Hirabayashi et al., Leukemia, 1 1(3):489-492, 1997.
Hoyes et al., Int. J. Radiat. Biol., 76:1435-1442, 2000.
Kaneda et al., Science, 243:375-378, 1989.
Kaplitt et al., Nat. Genet., 8:148-153, 1994.
Kato et al, J. Biol. Chem., 266:3361-3364, 1991.
Kessler et al., Proc. Natl. Acad. Sci. USA, 93:14082-14087, 1996.
Klein et al., Nature, 327:70-73, 1987.
Koeberl et al., Proc. Natl. Acad. Sci. USA, 94:1426-1431, 1997.
Levrero et al., Gene, 101:195-202, 1991.
Los et al., Mol. Biol. Cell, 13:978-988, 2002.
Mann et al., Cell, 33:153-159, 1983.
Mauch et al., Int. J. Radiat. Oncol. Biol. Phys., 31:1319-1339, 1995.
McCown et al., Brain Res., 713:99-107, 1996.
Mizukami et al., Virology, 217:124-130, 1996.
Nagafuji et al., Leuk. Lymphoma, 24:43-56, 1996.
Naito et al., Blood, 89:2060-2066, 1997.
Neta, and Okunieff, Semin. Radiat. Oncol., 6:306-320, 1996.
Nicolas and Rubenstein, In: Vectors: A survey of molecular cloning vectors and their uses, Rodriguez and Denhardt (Eds.), Stoneham: Butterworth, 493-513, 1988.
Nicolau and Sene, Biochim. Biophys. Acta, 721:185-190, 1982.
Nicolau et al., Methods Enzymol., 149:157-176, 1987.
Norbury and Hickson, Annu. Rev. Pharmacol. Toxicol., 41:367-401, 2001.
Okada et al., Blood, 80:3044-3050, 1992.
Paskind et al., Virology, 67:242-248, 1975.
Perales et al., Proc. Natl. Acad. Sci. USA, 91:4086-4090, 1994.
Perkins et al., J. Immunol., 138:466-469, 1987.
Ping et al., Microcirculation, 3:225-228, 1996.
Ploemacher et al., Int. J. Radiat. Biol., 61:489-499, 1992.
Poggi et al., Curr. Probl. Cancer, 25:334-411, 2001.
Potter et al., Proc. Natl. Acad. Sci. USA, 81:7161-7165, 1984.
Pruschy et al., Int. J. Radiat. Oncol. Biol. Phys., 49:561-567, 2001.
Radler et al., Science, 275:810-814, 1997.
Randall and Weissman, Blood, 89:3596-3606, 1997.
Renan, Radiother. Oncol., 19:197-218, 1990.
Ridgeway, In: Vectors: A survey of molecular cloning vectors and their uses, Rodriguez and Denhardt (Ed.), Stoneham:Butterworth, 467-492, 1988.
Rippe et al., Mol. Cell Biol., 10:689-695, 1990.
Roux et al., Proc. Natl. Acad. Sci. USA, 86:9079-9083, 1989.
Sambrook et al., In: Molecular Cloning: A Laboratory Manual, 2d Ed., Cold Spring Harbor Laboratory Press, Cold Spring Harbor, N.Y., 2001.
Samulski et al., J. Virol., 61(10):3096-3101, 1987.
Sane and Bertrand, Cancer Res., 59:3565-3569, 1999.
Seidita et al., Carcinogenesis, 21:2203-2210, 2000.
Shen and White, Adv. Cancer Res., 82:55-84, 2001.
Susin et al., J. Exp. Med., 189:381-394, 1999.
Temin, In: Gene Transfer, Kucherlapati (ed.), NY, Plenum Press, 149-188, 1986.
Tibbetts Cell, 12:243-249, 1977.
Tur-Kaspa et al., Mol. Cell Biol., 6:716-718, 1986.
Wagner et al., Proc. Natl. Acad. Sci. USA, 87(9):3410-3414, 1990.
Watt et al., Proc. Natl. Acad. Sci. USA, 83(2): 3166-3170, 1986.
Weiss and M.R. Landauer, Ann. NY Acad. Sci., 899:44-60, 2000.
White et al. J. Virol., 73(4):2832-2840, 1999.
WO 91/15577
WO 93/05071
WO 93/09135
WO 93/14777
WO 93/16710
WO 95/26958
Wong et al., Gene, 10:87-94, 1980.
Wu and Wu, Adv. Drug Delivery Rev., 12:159-167, 1993.
Wu and Wu, Biochem., 27:887-892, 1988.
Wu and Wu, J. Biol. Chem., 262:4429-4432, 1987.
Xiao et al., J. Virol., 70:8098-8108, 1996.
Yang et al., Proc. Natl. Acad. Sci USA, 87:9568-9572, 1990.
Young, In: Militory Radiobiology, Conklin and Walker (Eds.) Academic Press, Inc., Orlando, Fla., 6-191, 1987.
Zucali, Leuk. Lymphoma, 13:27-32, 1994.

1. A method of inhibiting apoptosis in a hematopoietic stem cell (HSC) comprising contacting said cell with a caspase inhibitor in an amount sufficient to inhibit apoptosis in said cell.

2. The method of claim 1, wherein apoptosis is induced by ionizing radiation.

3. The method of claim 2, wherein said HSC is contacted with said ionizing radiation before said caspase inhibitor.

4. The method of claim 3, wherein said HSC is contacted with said caspase inhibitor about 4 hours after receiving said ionizing radiation.

5. The method of claim 2, wherein said HSC is contacted with said ionizing radiation after said caspase inhibitor.

6. The method of claim 5, wherein said HSC is contacted with said caspase inhibitor about 2 hours before to receiving said ionizing radiation.

7. The method of claim 1, wherein said caspase inhibitor is contacted with said HSC more than one time.

8. The method of claim 2, wherein said caspase inhibitor is administered both prior to and after ionizing radiation is contacted with said HSC.

9. The method of claim 1, wherein said caspase inhibitor is z-VAD, BocD, LY333531, casputin, Ac-DQMD-CHO, CV-1013, VX-799, Ac-YVAD-CMK, IDN-5370, IDN-6556, IDN-6734, IDN-1965, IDN-1529, z-VAD-fmk, z-DEVD-cmk, Ac-YVAD-fmk, z-Asp-Ch2-DCB, Ac-IETD, Ac-VDVAD, Ac-DQMD, Ac-LEHD, or Ac-VEID.

10. The method of claim 1, wherein said HSC is contacted with a second agent.

11. The method of claim 10, wherein said second agent is a second caspase inhibitor.

12. The method of claim 10, wherein said second agent is a radioprotectant.

13. The method of claim 11, wherein said second agent is an anti-apoptotic protein, a cytokine or growth factor.

14. The method of claim 13, wherein said anti-apoptotic protein, cytokine or growth factor is expressed in said HSC from a recombinant expression vector.

15. The method of claim 14, wherein said expression vector is a viral vector.

16. The method of claim 14, wherein said expression vector is a non-viral vector.

17. A method of inhibiting radiation-induced injury in a subject comprising administering to said subject a caspase inhibitor in an amount sufficient to inhibit radiation-induced injury.

18. The method of claim 17, wherein said caspase inhibitor, is administered orally or by injection.

19. The method of claim 17, wherein said caspase inhibitor is z-VAD, BocD, LY333531, casputin, Ac-DQMD-CHO, CV-1013, VX-799, Ac-YVAD-CMK, IDN-5370 IDN-6556, IDN-6734, IDN-1965, IDN-1529, z-VAD-fmk, z-DEVD-cmk, Ac-YVAD-fmk, z-Asp-Ch2-DCB, Ac-IETD, Ac-VDVAD, Ac-DQMD, Ac-LEHD, or Ac-VEID.

20. The method of claim 17, further comprising administering to said subject a second agent.

21. The method of claim 20, wherein said second agent is a second caspase inhibitor, an anti-apoptotic molecule, a radioprotectant, a cytokine, or a growth factor.

22. The method of claim 20, wherein said second agent is an anti-apoptotic protein.

23. The method of claim 22, wherein the anti-apoptotic protein is selected from the group consisting of a p53 inhibitor, Bcl-X_{L}, Bcl-2, c-IAP1, c-IAP2, and XIAP.

24. The method of claim 20, wherein said second agent is a cytokine.

25. The method of claim 24, wherein said cytokine is IL-1, IL-2, IL-3, IL-4, IL-5, IL-6, or IL-7.

26. The method of claim 20, wherein said second agent is a growth factor.

27. The method of claim 26, wherein said growth factor is Flt3 ligand, c-Kit ligand, M-CSF, GM-CSF, G-CSF, VEGF, erythropoietin, or leukemia inhibitory factor.

28. The method of claim 20, wherein said second agent is a radioprotectant.

29. The method of claim 28, wherein said radioprotectant is amifostine, vitamin E, vitamin C, selenium, melatonin, 5-androstenediol, cucumin, α-phenyl-tert-butylnitrone, a flavinoid, or a nitroxide.

30. The method of claim 22, wherein said anti-apoptotic protein, cytokine or growth factor is expressed from a recombinant expression vector encoding said anti-apoptotic protein and an HSC-selective promoter.

31. The method of claim 17, wherein said caspase inhibitor is provided prior to exposure to radiation.

32. The method of claim 17, wherein said caspase inhibitor is provided following exposure to radiation.

33. The method of claim 32, wherein said caspase inhibitor is provided about four hours or less following exposure to radiation.

34. The method of claim 32, wherein said caspase inhibitor is administered more than once.

35. The method of claim 32, wherein said caspase inhibitor is provided via continuous infusion.

36. A method of screening a caspase inhibitor for its ability to inhibit radiation-induced injury comprising:
(a) providing a hematopoietic stem cell (HSC);
(b) contacting said HSC with a dose of ionizing radiation sufficient to induce apoptosis in said HSC;
(c) contacting said HSC with said caspase inhibitor; and
(d) assessing one or more apoptotic characteristics in said HSC,
wherein a reduction in the number or extent of apoptotic characteristics in said HSC, as compared to an HSC not treated with said caspase inhibitor, identifies said caspase inhibitor as an inhibitor of radiation-induced injury.

37. The method of claim 36, wherein said method comprises the use of multiple HSCs, and assessing comprises measuring the number of said HSCs undergoing apoptosis.

38. The method of claim 37, wherein assessing comprises TUNEL assay, Annexin V-7AAD or PI staining, sub G0/1 cell analysis, or caspase activity assay.

39. The method of claim 37, wherein assessing comprises flow cytometry that can discriminate between Lin⁻ Scal⁺ c-kit⁺, Lin⁻ Scal⁻ c-kit⁺, Lin⁻Scal⁻ c-kit⁻ cells.

40. The method of claim 36, wherein at least steps (b) and (c) are performed in vivo.

41. The method of claim 36, wherein said HSC is isolated and at least steps (b) and (c) are performed in vitro.

42. The method of claim 36, wherein said characteristics of apoptosis comprise Annexin-V staining, caspase activation, and/or DNA fragmentation.

43. The method of claim 36, further comprising contacting said HSC with a second agent.

44. The method of claim 43, wherein said second agent is selected from the group consisting of an anti-apoptotic molecule (a p53 inhibitor or an anti-apoptotic protein, such as Bcl-X_{L}, Bcl-2, c-LAP1, c-IAP2, and XIAP), a radioprotectant (amifostine, vitamin E, vitamin C, selenium, melatonin, 5-androstenediol, cucumin, α-phenyl-tert-butylnitrone, a flavinoid, or a nitroxide), a cytokine (IL-1, IL-2, IL-3, IL-4, IL-5, IL-6, IL-7), or a growth factor (Flt3 ligand, c-Kit ligand, M-CSF, GM-CSF, G-CSF, VEGF, erythropoietin, leukemia inhibitory factor).

45. The method of claim 36, further comprising assessing one or more apoptotic characteristics in an HSC not treated with said caspase inhibitor.

46. A composition comprising a radioprotectant and a second agent selected from the group consisting of an anti-apoptotic molecule (a p53 inhibitor or an anti-apoptotic protein, such as Bcl-X_{L}, Bcl-2, c-IAP1, c-IAP2, and XIAP), a radioprotectant (amifostine, vitamin E, vitamin C, selenium, melatonin, 5-androstenediol, cucumin, α-phenyl-tert-butylnitrone, a flavinoid, or a nitroxide), a cytokine (IL-1, IL-2, IL-3, IL-4, IL-5, IL-6, IL-7), or a growth factor (Flt3 ligand, c-Kit ligand, M-CSF, GM-CSF, G-CSF, VEGF, erythropoietin, leukemia inhibitory factor).
